(19) United States
(12) Patent Application Publication
(10) Pub. No.: US 2019/0202898 A9
(43) Pub. Date: Jul. 4, 2019

(54) AGLYCOSYLATED ANTI-C3b ANTIBODIES AND USES THEREOF

(71) Applicant: NovelMed Therapeutics, Inc., Cleveland, OH (US)

(72) Inventor: Rekha Bansal, Cleveland, OH (US)

(21) Appl. No.: 15/012,457

(22) Filed: Feb. 1, 2016

Related U.S. Application Data
(63) Continuation-in-part of application No. 14/994,993, filed on Jan. 13, 2016, now abandoned.
Application No. 14/994,993 is a continuation-in-part of application No. 13/646,286, filed on Oct. 5, 2012, now Pat. No. 9,745,367.
Application No. 14/994,993 is a continuation of application No. 14/390,645, filed on Oct. 3, 2014, now Pat. No. 9,243,060.
(60) Provisional application No. 61/619,860, filed on Apr. 3, 2012.

(51) Int. Cl. C07K 16/18

(52) U.S. Cl. CPC C07K 16/18; C07K 2317/41; C07K 2317/24; C07K 2317/524; C07K 2317/92; C07K 2317/51; C07K 2317/71; C07K 2317/76; C07K 2317/52

(57) ABSTRACT

An aglycosylated humanized anti-C3b (AAC3b) antibody or antigen binding fragment thereof includes a modification at a conserved N-linked site in the CH2 domains of an Fc portion of the antibody or antigen binding fragment thereof.

### RELATED APPLICATION

This application is a Continuation-in-Part of U.S. patent application Ser. No. 14/994,993, filed Jan. 13, 2016, which is a Continuation of U.S. patent application Ser. No. 14/390,645, filed Oct. 3, 2014, (Now U.S. Pat. No. 9,243,060), which is a National Phase Filing of PCT/US2013/034990, which claims priority to U.S. Provisional Application No. 61/619,860, filed Apr. 3, 2012, the subject matter of which are incorporated herein by reference in their entirety.

### BACKGROUND

The complement system is activated via three distinct pathways; the classical pathway (CP), the lectin pathway, and the alternative complement pathway (AP). The classical pathway (CP) is activated via antigen-antibody complexes. The lectin pathway is a variation of the classical pathway and the alternative pathway (AP) is activated by foreign material, artificial surfaces, dead tissues, bacteria, and dead yeast cells.

The classical complement pathway is important for host defense against pathogens. Activation of the classical pathway generates C3a, C4a, C5a and C5b-9 molecules, which activates a variety of cells in response to host defense. In pathological conditions, as a result of activation of the alternative pathway, anaphylatoxins C3a, C5a are formed and tissues damaging C5b-9 molecules, also known as the membrane attack complex (MAC), are formed. These molecules mediate inflammation via cellular activation and release of inflammatory mediators. In addition to its role as a lytic pore-forming complex, there is strong evidence that the deposition of sublytic MAC may play an important role in inflammation.

The alternative complement pathway is activated in pathological inflammation. Elevated levels of C3a, C5a, and C5b-9 have been found associated with multiple acute and chronic disease conditions. These inflammatory molecules activate neutrophils, monocytes and platelets. Therefore, inhibition of disease-induced AP activation is important for clinical benefit in the diseases where complement activation plays a role in disease pathology.

In addition to its essential role in immune defense, the complement system contributes to tissue damage in many clinical conditions. The activities included in the complement biochemical cascade present a potential threat to host tissue. An example includes the indiscriminate release of destructive enzymes possibly causing host cell lysis. Thus, there is a need to develop therapeutically effective complement inhibitors to prevent these adverse effects.

In a disease condition where AP activation contributes to disease pathology, elevated levels of C3a, C5a and C5b-9 molecules are found in serum, plasma, blood or other body fluids representative of the disease. Production and inhibition of each of these molecules via different mechanisms is important for disease pathology.

Based upon the available clinical and research data, it appears that in most acute and chronic settings, production of C3a and C5a is mediated by the activation of the complement pathways. Both of the anaphylatoxins C3a and C5a are known to activate leukocytes and platelets. A frequent indicator of cellular activation is the cellular expression of CD11b on leukocytes, and CD62P on platelets. The release of several inflammatory molecules is triggered by the platelet-leukocyte binding mediated by these activation markers. One result of such conjugate formation is the removal of platelets from the circulation, a phenomenon that can contribute to the development of thrombocytopenia.

### SUMMARY

Embodiments described herein relate to an aglycosylated or aglycosyl anti-C3b (AAC3b) antibody or antigen (i.e., C3b) binding fragment thereof that binds C3b and inhibits alternative pathway activation and C3 dependent complement activation, and particularly relates to an aglycosylated or aglycosyl humanized anti-C3b antibody that binds C3b and inhibits C3 dependent complement activation. The AAC3b antibody or antigen binding fragment thereof can be used to treat a complement-mediated disease in a subject in need thereof.

In some embodiments, the AAC3b antibody or antigen binding fragment thereof includes a modification at the conserved N-linked site of the CH2 domain of the Fc portion of the antibody. The modification can include a mutation in the heavy chain glycosylation site that prevents glycosylation at the site. In some embodiments, the modification includes a mutation of N298Q (N297 using EU Kabat numbering). In other embodiments, the modification includes a mutation of N298A (N297 using EU Kabat numbering). In still other embodiments, the modification includes the removal of the CH2 domain glycans. The modification can prevent glycosylation at the CH2 domain.

In some embodiments, the AAC3b antibody or antigen binding fragment thereof can include a humanized heavy chain aglycosylated region having an amino acid sequence selected from the group consisting of: SEQ ID NOs: 24-57.

In some embodiments, the AAC3b antibody or antigen binding fragment thereof does not bind to an Fc effector receptor and/or cause cellular lysis.

In other embodiments, the AAC3b antibody or antigen binding fragment thereof is selected from the group consisting of: monoclonal antibodies, polyclonal antibodies, murine antibodies, chimeric antibodies, primatized antibodies, and humanized antibodies.

In some embodiments, the AAC3b antibody or antigen binding fragment thereof is selected from the group consisting of: multimeric antibodies, heterodimeric antibodies, hemidimeric antibodies, tetravalent antibodies, bispecific antibodies, Fab, Fab′, Fab′2, F(v) antibody fragments, and single chain antibodies or derivatives thereof.

In other embodiments, the AAC3b antibody or antigen binding fragment thereof can be an aglycosylated humanized antibody of the murine monoclonal antibody produced by a hybridoma cell deposited under ATCC Accession No. PTA-8806. In some embodiments, the AAC3b antibody or antigen binding fragment thereof can have a heavy chain variable domain including 3CDRs having the amino acid sequences of SEQ ID NO: 2, SEQ ID NO: 3 and SEQ ID NO: 4 and a light chain variable domain including 3CDRs having amino acid sequences of SEQ ID NO: 19, SEQ ID NO: 20, and SEQ ID NO: 21.

In some embodiments, the heavy chain variable domain can have an amino acid sequence at least 90% identical to SEQ ID NO: 1. For example, the heavy chain variable domain can have an amino acid sequence selected from the group consisting of SEQ ID NO: 6, SEQ ID NO: 7; SEQ ID NO: 8; SEQ ID NO: 9; SEQ ID NO: 10; SEQ ID NO: 11; SEQ ID NO: 12, SEQ ID NO: 13; SEQ ID NO: 14; SEQ ID NO: 15; SEQ ID NO: 16; and SEQ ID NO: 17. In other embodiments, the light chain variable domain can have an amino acid sequence selected from the group consisting of SEQ ID NO: 18; SEQ ID NO: 22; and SEQ ID NO: 58.

In other embodiments, the AAC3b antibody, antigen binding fragment thereof, or pharmaceutical composition thereof can be administered to a subject by injection, intravenously, subcutaneously, intravitreally, intraperitoneally, intramuscularly, intramedullarily, intraventricularly, intraepidurally, intraarterially, intravascularly, intra-articularly, intra-synovially, intrasternally, intrathecally, intrahepatically, intraspinally, intratumorly, intracranially, enteral, intrapulmonary, transmucosal, intrauterine, sublingual, or locally at sites of disease pathology.

Other embodiments relate to a method of inhibiting alternative complement pathway in a subject in need thereof by administering to the subject an inhibiting amount of an AAC3b antibody, antigen binding fragment thereof, or pharmaceutical composition thereof. The AAC3b antibody or antigen binding fragment thereof includes a mutation of one the asparagine residue (N297 using EU Kabat numbering) at the conserved N-linked sites in the CH2 domains of the Fc portion of the antibody. The mutation prevents glycosylation at the site and does not contribute to the binding and functional properties of the antibody.

In some embodiments, the AAC3b antibody or antigen binding fragment thereof can display similar characteristics for function and affinity binding to C3b as a murine anti-C3b antibody (AC3b antibody), such as an AC3b antibody produced by the hybridoma cell line deposited under ATCC Accession No. PTA-8806. For example, the AAC3b antibody or antigen binding fragment thereof can inhibit binding of C3b to Factor B at the same concentration as the AC3b antibody. The AAC3b antibody can also specifically bind to the same epitope as the AC3b antibody or compete with AC3b antibody for C3b binding.

In some embodiments, the AAC3b antibody or antigen binding fragment thereof can include at least one of the following properties: specifically bind C3b and prevent formation of C3a and C3b; specifically bind C3b and prevent formation of C5a and C5b, specifically bind C3b and prevent formation of SC5b-9, C5b-6, C5b-7, C5b-8, and C5b-9, specifically bind C3b and prevent formation and deposition of C3b, specifically bind C3b and prevent formation and deposition of PC3b, specifically bind C3b and prevent formation and deposition of PC3bBb, specifically bind C3b and prevent formation and deposition of (P)n(C3b)n(Bb)n where n is equal to any value between 1 to 10, specifically bind C3b and prevent activation of neutrophils, monocytes, and platelets via the inhibition of AP, specifically bind C3b and prevent formation of various cytokines including VEGF and IL-1, specifically bind C3b and prevent lysis of erythrocytes that do lack or do not carry human CD55 or CD59, or specifically bind C3b and prevent lysis of platelets.

In other embodiments, the AAC3b antibody or antigen binding fragment thereof can be conjugated to a detectable marker, therapeutic agent, imaging agent, or radionuclide. The detectable marker can be, for example, a radioactive isotope, enzyme, dye, or biotin. The therapeutic agent can be, for example, a radioisotope, radionuclide, toxin, toxoid or chemotherapeutic agent. The imaging agent can be a labeling moiety, biotin, a fluorescent moiety, a radioactive moiety, a histidine tag, or a peptide tag.

Still other embodiments relate to a pharmaceutical composition that includes an AAC3b antibody and a pharmaceutically acceptable carrier. The AAC3b antibody or antigen binding fragment thereof binds C3b and inhibits alternative pathway activation and particularly C3 dependent complement activation. The AAC3b antibody or antigen binding fragment thereof can be used to treat a complement-mediated disease in a subject in need thereof.

The AAC3b antibody or antigen binding fragment thereof can include a modification at the conserved N-linked site in the CH2 domains of the Fc portion of said antibody. The modification can include a mutation in the heavy chain glycosylation site that prevents glycosylation at the site. In some embodiments, the modification includes a mutation of N298Q (N297 using EU Kabat numbering). In other embodiments, the modification includes a mutation of N298A (N297 using EU Kabat numbering). In still other embodiments, the modification includes the removal of the CH2 domain glycans. The modification can prevent glycosylation at the CH2 domain.

In some embodiments, the pharmaceutical composition can further include an immunosuppressive or immunomodulatory compound. The pharmaceutical composition can also include a buffer at a pH 6 to 6.5. The AAC3b antibody or antigen binding fragment thereof can be provided in the formulation in the range of about 20 mg/mL to about 200 mg/mL, for example, about 50 mg/ml to about 100 mg/ml.

Other embodiments relate to a method for ameliorating complement-mediated diseases in a subject by administering to the subject a therapeutically effective amount of an AAC3b antibody or antigen binding fragment thereof. The AAC3b antibody or antigen binding fragment thereof can include a modification at the conserved N-linked site in the CH2 domains of the Fc portion of the antibody. The modification can include a mutation in the heavy chain glycosylation site that prevents glycosylation at the site. In some embodiments, the modification includes a mutation of N298Q (N297 using EU Kabat numbering). In other embodiments, the modification includes a mutation of N298A (N297 using EU Kabat numbering). In still other embodiments, the modification includes the removal of the CH2 domain glycans. The modification can prevent glycosylation at the CH2 domain.

The AAC3b antibody, antigen binding fragment thereof, or pharmaceutical composition thereof can be administered to the subject in any manner that is medically acceptable, such as by oral, nasal, ophthalmic, rectal, and topical routes. For example, the AAC3b antibody, antigen binding fragment thereof, or pharmaceutical composition thereof can be administered, orally in the form of capsules, tablets, aqueous suspensions or solutions, topically by application of a cream, ointment or the like, by inhalation through the use of a nebulizer, a dry powder inhaler or a metered dose inhaler, or by sustained release administration.

In some embodiments, the AAC3b antibody, antigen binding fragment thereof, or pharmaceutical composition thereof can be administered to the subject in multiple doses per day, repeatedly at intervals ranging from each day to every other month, or at intervals for as long a time as medically indicated, ranging from days or weeks to the life of the subject.

Still other embodiments relate to a method for inhibiting alternative complement pathway but not activating the classical pathway in a subject by administering to the subject a therapeutically effective amount of an AAC3b antibody or antigen binding fragment thereof. The AAC3b antibody or antigen binding fragment thereof can include a modification at the conserved N-linked site in the CH2 domains of the Fc portion of said antibody. The modification can include a mutation in the heavy chain glycosylation site that prevents glycosylation at the site and C1Q binding so that the AAC3b antibody or antigen binding fragment thereof does not activate the classical complement pathway.

In some embodiments, the AAC3b antibody or antigen binding fragment thereof does not bind C1Q and prevents C1Q mediated activation of the classical pathway, does not block classical pathway activation, and/or does not participate in the classical pathway activation.

Other embodiments described herein relate to a method for inhibiting alternative complement pathway but not activating the Fc effector in a subject by administering to the subject a therapeutically effective amount of an AAC3b antibody or antigen binding fragment thereof. The AAC3b antibody or antigen binding fragment thereof can include a modification at the conserved N-linked site in the CH2 domains of the Fc portion of said antibody. The modification can include a mutation in the heavy chain glycosylation site that prevents glycosylation at the site. In some embodiments, the modification includes a mutation of N298Q (N297 using EU Kabat numbering). In other embodiments, the modification includes a mutation of N298A (N297 using EU Kabat numbering). The mutation can prevent binding to the Fc receptors on a variety of cells and the AAC3b antibody or antigen binding fragment thereof does not activate the cells via Fc activation.

In some embodiments, the AAC3b antibody or antigen binding fragment thereof does not bind to Fc receptors selected from the group comprising; CD16a, CD16b, CD32a, CD32b, CD32c, and CD64 and therefore prevents Fc activation on cells. The Fc receptors, CD16a, CD16b, CD32a, CD32b, CD32c, and CD64, can be present on cells selected from the group comprising Neutrophils, monocytes, platelets, T lymphocytes, NK cells, basophils, and eosinophils, and activation of such cells is prevented by administering the AAC3b antibody or antigen binding fragment thereof to the cells. The cells can also cause inflammatory and thrombotic events, which are prevented with administration of the AAC3b antibody or antigen binding fragment thereof to the cells.

Still other embodiments relate to a method for inhibiting, treating, preventing complement-mediated disease in a subject by administering to the subject a therapeutically effective amount of the AAC3b antibody or antigen binding fragment thereof to the subject. The complement-mediated disease can be selected from the group consisting of: inflammatory disorders, Extracorporeal Circulation Disorders, Cardiovascular Disorders, Musculoskeletal Disorders, Ocular Disorders, Transplantation disease Disorders, Hemolytic Disorders, Repiratory Disorders, Neurological Disorders, Trauma-induced Disorders, Renal Disorders, Dematological Disorders, Gastrointestinal Disorders, Endocrine Disorders, Reproduction and urogenital diseases and disorders, Reperfusion Injury Disorders.

Other embodiments relate to a method of imaging cells, organs, tissues in a subject that express the antigen C3b (the immunogen of the Anti-C3b antibody) or its fragments that is specifically recognized by the AAC3b antibody or AC3b antibody comprising the steps of: (a) administering to the subject an effective amount of an imaging composition comprising the AAC3b antibody, AC3b antibody, or antigen binding fragment thereof under conditions permitting the formation of a complex between the AAC3b antibody, AC3b, or antigen binding fragment thereof and the protein on the surface of cells, tissues, or organs; and (b) imaging any antibody/protein complex or antibody derivative/complex formed, thereby imaging disease cells in the subject.

Still other embodiments relate to a method for detecting the presence of C3b positive cells in a subject that express C3b that is specifically recognized by the AAC3b antibody or AC3b antibody comprising the steps of: (a) administering to the subject an effective amount of an imaging agent comprising the AAC3b antibody, AC3b antibody, or antigen binding fragment thereof under conditions permitting the formation of a complex between the antibody or antibody derivative and the protein; (b) clearing any unbound imaging agent from the subject; and (c) detecting the presence of any antibody/protein complex or antibody derivative/complex formed, the presence of such complex indicating the presence of disease cells in the subject

### BRIEF DESCRIPTION OF THE DRAWINGS

FIG. 1 illustrates a plot showing that the AAC3b antibody binds substrate-bound C3b with high affinity of 100 pM using an ELISA assay. ELISA wells were coated with Factor C3b at a fixed concentration. AAC3b at various concentrations in solution were allowed to bind and the data was fitted using Origin graphing program.

FIG. 2 illustrates a plot showing that the AAC3b antibody inhibits alternative pathway dependent hemolysis of erythrocytes in 90% NHS. AAC3b inhibits hemolysis in a dose-dependent manner with approximately 50 nM antibody required to neutralize the C3b in undiluted normal human serum. In this experiment, various concentrations of AAC3b antibody were added to undiluted human serum and the mixture was subjected to AP hemolysis. The data demonstrates that AP Hemolysis is inhibited in human serum.

FIG. 3 illustrates a plot showing AAC3b antibody inhibits AP hemolysis without inhibiting CP hemolysis. This study was conducted in whole blood. Whole human blood from six individuals was treated with the AAC3b antibody. As shown the AAC3b antibody did not inhibit CP but AP is inhibited as usual.

FIG. 4 illustrates a plot showing that AAC3b antibody does not bind C1Q. In this experiment various concentrations of Normal human serum was incubated over AAC3b coated plates. Avastin was used as a positive control and, as expected, binds C1Q present in serum. In contrast, AAC3b antibody has no binding suggesting that the AAC3b antibody has reduced C1Q binding and therefore lack of CP activation and less effector function.

FIG. 5 illustrates a plot showing the results of C3 convertase formation. AAC3b antibody inhibits C3 convertase formation in a dose-dependent formation. Inhibition of properdin binding in a dose-dependent manner reflects the inhibition of C3 convertase formation (PC3bBb). ELISA plates were coated with LPS and incubated in Normal human serum at 10% in AP buffer.

FIG. 6 illustrates a plot showing the results of C3 convertase formation. AAC3b antibody inhibits C3 convertase formation in a dose-dependent formation. Inhibition of C3b deposition on the LPS in a dose-dependent manner reflects the inhibition of C3 convertase formation (PC3bBb). ELISA plates were coated with LPS and incubated in Normal human serum at 10% in AP buffer.

FIG. 7 illustrates a plot showing the results of C3 convertase formation. AAC3b antibody inhibits C3 convertase (PC3bBb) formation in a dose-dependent formation. Inhibition of Bb formation is inhibited in a dose-dependent manner reflects the inhibition of C3 convertase formation (PC3bBb). ELISA plates were coated with LPS and incubated in Normal human serum at 10% in AP buffer. The Bb was detected with an anti-Factor B antibody.

FIG. 8 illustrates a plot showing the results from a convertase formation assay. In this experiment, detection of C5b indicates the presence of MAC (C5b-9). The data shows that increasing concentrations of AAC3b antibody inhibits C5b formation.

FIG. 9 illustrates a plot showing the results from a convertase formation assay. In this experiment, C5b-9 was detected with neo anti-MAC antibody which identifies deposited MAC (C5b-9). The data shows that increasing concentrations of AAC3b antibody inhibits MAC formation.

FIG. 10 illustrates a plot showing unlabeled AC3b antibody competes with the labeled AC3b antibody for C3b binding. ELISA plates were coated with C3b. Varying concentrations of unlabeled AC3b antibody were added to the fixed concentration of labeled AC3b antibody. Following a typical competition assay method. We determined that unlabeled antibody competes with the labeled antibody in a dose dependent manner. Therefore both the labeled AC3b antibody and unlabeled AC3b antibody share the same epitope on C3b.

FIG. 11 illustrates plots showing aglycosylated Fc does not bind to CD16a, CD16b or CD32a. BIACORE methods were used

FIG. 12 illustrates plots showing aglycosylated Fc does not bind to CD32b and CD32c. It also shows substantially reduced binding to FcRn and CD64 compared to control glycosylated IgG. BIACORE methods were applied.

FIG. 13 illustrates various alternative constructs of the HC variable region of AAC3b antibody can be made using the consensus sequence (SEQ ID NO: 5) and making the point substitutions shown.

FIG. 14 illustrates schematically activation of the alternative pathway (AP) produces two potent anaphylatoxins; C3a and C5a. These anaphylatoxins activate a variety of cells. Activated cells release various inflammatory mediators that have been shown to be involved in disease pathology. Use of AAC3b is expected to prevent the formation of C3a/C3b, C5a/C5b, and MAC and therefore provide therapeutic benefit in diseases mediated or associated with complement activation.

FIG. 15 lists the amino acid sequences of the AAC3b antibody heavy chain and light chain CDRs (SEQ ID NOs: 2-4 and SEQ ID NOs: 19-21).

FIG. 16 lists the amino acid sequences of humanized heavy chain variable region for AAC3b antibodies (SEQ ID NOs: 1, and 6-17).

FIGS. 17, 18, 19, and 20 list amino acid sequences of heavy chain constant regions with aglycosylation (SEQ ID NOs: 23-57).

FIG. 21 lists amino acid sequences of the light chain variable and constant regions (SEQ ID NOs: 18, 22, and 58).

### DETAILED DESCRIPTION

The following definitions are provided in order to provide clarity with respect to the terms as they are used in the specification and claims, in order to describe the present invention.

The term “alternative pathway” refers to complement activation, which has traditionally been thought to arise from spontaneous proteolytic generation of C3b from complement factor C3 triggered, for example, by zymosan from fungal and yeast cell walls, lipopolysaccharide (LPS) from Gram-negative outer membranes, and rabbit erythrocytes, as well as from many pure polysaccharides, rabbit erythrocytes, viruses, bacteria, animal tumor cells, parasites and damaged cells.

The term “antibody” encompasses antibodies and antibody fragments, which specifically bind to C3b or its polypeptides or portions, in which the antibody is derived from any antibody-producing mammal (e.g., a mouse, a rat, a rabbit, or a primate, including a human). Exemplary antibodies include polyclonal, monoclonal and recombinant antibodies; multi-specific antibodies (e.g., bi-specific antibodies), humanized antibodies; murine antibodies, chimeric (i.e., mouse-human, mouse-primate, primate-human), monoclonal antibodies, and anti-idiotype antibodies, as well as de-immunized antibodies, and may be any intact molecule or fragment thereof.

The term “antibody fragment” refers to a portion derived from or related to a full-length anti-C3b antibody, generally including the antigen binding or variable region thereof. Illustrative examples of antibody fragments include Fab, Fab′, F(ab)2, F(ab′)2 and Fv fragments, scFv fragments, diabodies, linear antibodies, single-chain antibody molecules and multispecific antibodies formed from antibody fragments.

The term “antigen binding fragment” refers to a fragment or fragments of a C3b antibody that contain the antibody variable regions responsible for antigen binding. Fab, Fab′, and F(ab)2 lack the FC regions. Antigen-binding fragments can be prepared from full-length antibody by protease digestion. Antigen-binding fragments may be produced using standard recombinant DNA methodology by those skilled in the art.

The term complementarity-determining region (“CDR”) refers to a specific region within variable regions of the heavy and the light chain. Generally, the variable region consists of four framework regions (FR1, FR2, FR3, FR4) and three CDRs arranged in the following manner: NH2-FR1-CDR1-FR2-CDR2-FR3-CDR3-FR4-COOH. The term “framework regions” refers to those variable domain residues other than the CDR residues herein defined.

The term “competitively inhibits” refers to competitive inhibition of binding of a isolated antibody or antigen binding fragment thereof to C3b by any other molecule.

The term “C3b inhibitory agent” refers to any agent that binds to or interacts with C3b and effectively inhibits C3b-dependent complement activation, including anti-C3b antibodies and C3b antigen binding fragments thereof, natural and synthetic peptides. C3b inhibitory agents useful in the methods described herein may reduce C3b-dependent complement activation, therefore all activation, by greater than 20%. In one embodiment, the C3b inhibitory agent reduces complement activation by greater than 90%.

A “chimeric antibody” is a recombinant protein that contains the variable domains and complementarity-determining regions derived from a non-human species (e.g., rodent) antibody, while the remainder of the antibody molecule is derived from a human antibody.

The term “classical pathway” refers to both (1) complement activation of the C1-complex triggered by an antibody bound to a foreign particle and requires binding of the recognition molecule C1q, and also to (2) complement activation that occurs via antigen-antibody complex formation.

A “humanized antibody” is a chimeric antibody that comprises a minimal sequence conforming to specific complementarity-determining regions derived from non-human immunoglobulin that is transplanted into a human antibody framework. Humanized antibodies are typically recombinant proteins in which only the antibody complementarity-determining regions are of non-human origin.

The term “lectin pathway” refers to complement activation that occurs via the specific binding of serum and non-serum carbohydrate-binding proteins including mannan-binding lectin (MBL) and the ficolins.

The terms “treatment,” “treating,” and the like, refer to obtaining a desired pharmacologic, biologic, and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The “membrane attack complex” (“MAC”) refers to a complex of the five terminal complement components (C5-C9) that inserts into and disrupts membranes. MAC can also be referred to as C5b-9.

The term “complement-mediated diseases” refers to diseases where one or more of complement activation products have been found elevated and or associated with tissue, bodily fluids, and organs.

The term “Fc effector” refers to activation of a variety of cells to release potent inflammatory mediators. Fc Effector functions provide positive benefit in healthy subjects. Unnecessary Fc effectors can cause chaos in the body and can lead to significant inflammatory response and activation of inflammatory cells. Fc effector response occurs when Fc portion of the antibody binds Fc receptors. CD16a, CD16b, CD32a, CD32b, CD32c if bound the therapeutic/diagnostic antibody can turn on the signal for a cytokine storm by activation of neutrophils, monocytes, platelets, NK cells, T lymphocytes etc. Such activation can not only generate a cytokine storm but can also cause thrombotic events by non-specific activation of platelets and erythrocytes. The AAC3b antibody appears to have low to no binding to these receptors and therefore would be a therapeutic without Fc effector function.

The term “C1Q binding” refers C1q binding to the antibody Fc region which can initiate the activation of the classical pathway. By removing the glycosylation, AAC3b binding to C1Q was reduced.

The terms aglycosylated or aglycosyl antibodies (e.g., AAC3b) refers to antibodies that are aglycosylated. Human antibodies are generally glycosylated naturally at asparagine residues. The antibodies can be aglycosylatd by single point mutations. Aglycosylation reduces C1Q interaction and provides the antibody with reduced Fc effector functions. Aglycosylation is generally introduced at the N297 position of the CH2 region. However, because of the varying lengths of the CDRs, the position of asparagines within the CH2 may change a bit. Irrespective of the exact position, if the “N297” is changed to Q (Glutamine) or any other residue such as “A (Ala)”, an aglycosylated antibody can be generated. Other means of making AAC3b aglycosylated can be proposed, such as removal of CH1 and CH2, removal of CH2, and or other point mutations that can cause aglycosylation.

The term “subject” refers to all mammals, including, but not limited to, dogs, cats, horses, sheep, goats, cows, rabbits, pigs, humans, non-human primates, and rodents. In studies where animals are used as models to address a disease, the term subject has been used. The term subject has also been used in case of human when the drug is said to be administered in humans.

The terms “C3b and fragments” refers to C3b which is made by the cleavage of C3 into C3b and C3a. C3b is known to deposit on tissues and cells. C3b can further degrade into iC3b, C3c, C3dg, and C3d. One or more of these fragments have been found associated in disease pathology and therefore one can predict that complement activation has occurred. Since C3 is part of the alternative complement pathway, it is reasonable to believe that pathologies where one or more of these fragments are found deposited would be treatable by the AAC3b antibodies.

Embodiments described herein relate to aglycosylated or aglycosyl anti-C3b (AAC3b) antibodies and antigen binding fragments thereof with reduced effector functions and to the use of such antibodies and antigen binding fragments thereof to inhibit alternative pathway complement activation and to treat complement-mediated diseases. The mechanism of action of glycosylated antibodies in treating complement-mediated diseases in vivo can be difficult to delineate as glycosylation can cause complement fixation and Fc effector function. In contrast, the mechanism of action of AAC3b antibodies is elucidated through the use of an AAC3b antibody in which Fc effector function has been reduced by a modification of the conserved N-linked site in the CH2 domains of the Fc dimer, leading to “aglycosyl” anti-C3b antibodies. Examples of such modifications include mutation of the conserved N-linked site in the CH2 domains of the Fc dimer, removal of glycans attached to the N-linked site in the CH2 domains and prevention of glycosylation.

To address whether the binding affinity and activity of AAC3b antibody is influenced by Fc effector interactions, murine anti-C3b (AC3b) antibody and AAC3b were tested with regard to their ability to bind C3b and block AP activation in vitro and whole blood. The results demonstrate that AC3b and AAC3b are comparable for C3b binding and AP inhibition.

Because the AAC3b antibodies described herein are characterized by diminished effector function, these antibodies are particularly desirable for use in subjects where the undesirable thrombo-embolitic, Fc effector response and complement fixation activities are to be removed. Additionally, the diminished Fc effector function of the AAC3b antibodies may further reduce the unwanted activation of T-lymphocytes, NK cells, monocytes/macrophages, neutrophils, erythrocytes and platelets as all these cells bear Fc receptors.

In some embodiment, the AAC3b antibody or antigen binding fragment thereof can include a modification at the conserved N-linked site in the CH2 domains of the Fc portion of the antibody. The modification can include a mutation in the heavy chain glycosylation site that prevents glycosylation at the site. In some embodiments, the modification includes a mutation of N298Q (N297 using EU Kabat numbering). In other embodiments, the modification includes a mutation of N298A (N297 using EU Kabat numbering). In still other embodiments, the modification includes the removal of the CH2 domain glycans. The modification can prevent glycosylation at the CH2 domain. In some embodiments, the AAC3b antibody or antigen binding fragment thereof does not bind to an Fc effector receptor and/or cause cellular lysis.

In other embodiments, the AAC3b antibody or antigen binding fragment thereof can include a humanized heavy chain aglycosylated region having an amino acid sequence selected from the group consisting of: SEQ ID NOs: 24-57.

In some embodiments, the AAC3b antibody or antigen binding fragment thereof can be an aglycosylated humanized antibody of the murine monoclonal antibody produced by a hybridoma cell deposited under ATCC Accession No. PTA-8806. The AAC3b antibody or antigen binding fragment thereof can have a heavy chain variable domain including 3CDRs having the amino acid sequences of SEQ ID NO: 2, SEQ ID NO: 3 and SEQ ID NO: 4 and a light chain variable domain including 3CDRs having amino acid sequences of SEQ ID NO: 19, SEQ ID NO: 20, and SEQ ID NO: 21.

In some embodiments, the heavy chain variable domain can have an amino acid sequence at least 90% identical to SEQ ID NO: 1. For example, the heavy chain variable domain can have an amino acid sequence selected from the group consisting of SEQ ID NO: 6, SEQ ID NO: 7; SEQ ID NO: 8; SEQ ID NO: 9; SEQ ID NO: 10; SEQ ID NO: 11; SEQ ID NO: 12, SEQ ID NO: 13; SEQ ID NO: 14; SEQ ID NO: 15; SEQ ID NO: 16; and SEQ ID NO: 17. In another embodiment, the light chain variable domain can have an amino acid sequence selected from the group consisting of SEQ ID NO: 18; SEQ ID NO: 22; and SEQ ID NO: 58.

In some embodiments, the AAC3b antibody or antigen binding fragment thereof can display similar characteristics for function and affinity binding to C3b as a murine anti-C3b antibody (AC3b antibody), such as an AC3b antibody produced by the hybridoma cell line deposited under ATCC Accession No. PTA-8806. For example, the AAC3b antibody or antigen binding fragment thereof can inhibit binding of C3b to Factor B at the same concentration as the AC3b antibody. The AAC3b antibody can also specifically bind to the same epitope as the AC3b antibody or compete with AC3b antibody for C3b binding.

The antibody can be, for example, a chimeric antibody, humanized antibody, human antibody, a humanized antibody or a chimeric antibody. The CDRs within the variable region may be 90% similar to about 99% similar.

In some embodiments AAC3b antibodies described herein can recognize C3b with high affinity without any change in the functional activity. The AAC3b and AC3b antibodies are capable of inhibiting the interaction between C3b and factor B. The AAC3b and AC3b antibodies do not inhibit properdin binding to C3b. Both antibodies can demonstrate comparable activity in a variety of alternative complement assays shown in the examples.

Another aspect relates to antibodies that bind to the same epitope on C3b as the antibodies described herein. Such antibodies can be identified based on their ability to cross-compete with or competitively inhibit anti-C3b antibodies or antigen binding fragment thereof in standard C3b binding assays.

For example as shown in FIG. 22, the binding of labeled anti-C3b antibody can compete with the unlabeled antibody or any other anti-C3b, which share the epitope with the antibodies described herein. The other anti-C3b antibodies can be humanized, mouse, fully human and/or may have any other format. The ability to block or compete with the antibodies described herein indicates that a C3b-binding antibody being examined binds to the same or similar epitope where the antibodies described herein bind. Several type of competition assays have been used by those skilled in the art. Competitive inhibition is measured by determining the amount of label bound to the solid surface or cells in the presence of antibodies being examined.

As these anti-C3b antibodies bind the substrate-bound C3b as shown in multiple ELISA assays, the antibody of the invention can detect C3b bound to cells, tissues, and substrate and therefore can be used in diagnostic procedures provided an appropriate label is chosen for detection. The label can be biochemical, radioactive, or PET or those well known in the art.

In some embodiments, the aglycosylated anti-C3b antibodies described herein are produced in a CHO cell-line by inserting the gene for the aglycosylated antibody. Other cell lines known in the art may also be used. Technological advancement can provide advanced methods of stable cell line production that are suited for drug production for use in vivo.

In another embodiment, the aglycosylated anti-C3b antibodies are able to associate with C3b in a manner that blocks, directly or indirectly the activation of C3b-bearing cells.

In some embodiments, the AAC3b antibodies or antigen binding fragments thereof described herein can be used in a method of treating or preventing, in a subject, an alternative pathway-dependent condition or disease, by administering to the subject the AAC3b antibodies or antigen binding fragments thereof, at an amount effective to inhibit AP activation in the subject and thereby treat or prevent the alternative pathway-dependent condition or disease.

In other embodiments, the AAC3b antibodies or antigen binding fragments thereof described herein can be used in a method of diagnosing, in a subject, alternative pathway-dependent condition or disease. The method can include administering to the subject an AAC3b antibody or antigen binding fragment thereof at an amount effective to bind surface bound C3b in the subject and thereby diagnosing alternative pathway-dependent condition or disease.

Still other embodiments relate to a method of inhibiting the adverse effects of Alternative Pathway (AP)-dependent complement activation in a living subject. The method includes administering to a subject in need thereof, an amount of the AAC3b antibodies or antigen binding fragments thereof effective to inhibit AP-dependent complement activation.

Other embodiments described relate to compositions for inhibiting alternative pathway dependent activation that include a therapeutically effective amount of the AAC3b antibodies or antigen binding fragments thereof and a pharmaceutically acceptable carrier. Such compositions can be beneficial in treating complement-mediated diseases where at least one of the following components of the alternative complement system have been identified in the human or animal subjects in disease condition, clinical trial, tissue/bodyfluid analysis or during animal studies. Such components are listed here for reference; C3a, C3a, C5a, C5b, sC5b-9, C5b-9, lack of CD55, lack of CD59, SC5b-9 and one or more cytokines.

The role of the alternative pathway in complement-mediated diseases is well documented. The classical pathway is required for host defense and must remain silent. The AP is triggered by damaged cells and tissue. AP is triggered by tissue damage. The AP consists of specific plasma proteins including complement Factors B, D, and P (Properdin). The C3 convertase of the AP cleave C3 into C3a and C3b. Likewise, C5 convertase cleaves C5 into C5a and C5b. The C5b molecules initiate the formation of membrane-attack complex (MAC, C5b-9). Formation of MAC causes further damage to tissues and organs via complement mediated attack on cell membranes. Several complement proteins, including sC5b-9 and C5b-9, have been found to be associated with several acute and chronic diseases. Histopathological studies have shown that there is an infiltration of inflammatory cells, including macrophages and lymphocytes, into the lesions that arise from disease exacerbation and progression. Complement protein deposition has also been identified. Elevated levels of complement proteins several knockout studies have further clarified the role of AP in complement-mediated diseases. Completion of the AP is indicated by the formation and deposition of C5b-9. Such molecules can activate cells, cause apoptosis and complete tissue injury leading to significant clinical symptoms. Currently there is much need to find a high affinity, target specific molecule with reduced effector function. Since classical pathway is required for host defense, the CP must remain silent and must remain unaffected by the drug. Thus AP inhibitors are an unmet need; AP activation produces two potent inflammatory molecules C3a and C5a which appear to orchestrate the inflammatory response leading to significant clinical pathology in human and animal subjects.

C3a and C5a Driven Inflammation—C3a and C5a bind to their respective receptors on neutrophils, monocytes, and platelets and activate these cells to produce inflammatory mediators. These inflammatory mediators further promote the inflammatory response. More specifically, C3a activates monocytes and lymphocytes, resulting in the release TNF-alpha, IL-1 alpha, VEGF, PDGF, prostaglandins, histamine, IL-6 and IL-8, from the activated cells. These agents have been implicated in a wide variety of disease pathologies ranging from arthritis to hemolytic blood disorders. Thus, C3a plays important roles in a variety of clinical situations. Likewise, C5a can up-regulate cell adhesion, initiate the release VEGF and induce lysosomal enzyme and free radical release from both neutrophils and monocytes. Activated complement byproducts C3a and C5a have been found to be present in drusen deposits.

C5b-9 and sC5b-9—The terminal AP activation byproducts sC5b-9 and C5b-9 (MAC) have been found to be present in disease tissues. Deposition of MAC on marks the onset of disease initiation and progression. Substantial MAC formation can directly cause cell death which results in tissue atrophy. However, even lesser, sublytic, concentrations of MAC can activate cell proliferation and migration, modulate cell functions, and induce inflammation. In PNH deposition of MAC can cause visual lysis of cells such as erythrocytes. Complement-mediated Diseases lists all diseases where complement components have been found in disease. Elevated levels of C3a, C3b, C5a, C5b, iC3b, C3dg, C3c, cytokines, growth factors, and MAC are all indicative of complement activation and therefore, AAC3b like molecules could provide therapeutic benefit to those suffering from diseases.

Complement-mediated diseases can include, for example, Inflammatory bowel disease, Rheumatoid arthritis, Rod-cone dystrophies, Acute lung injury, Acute respiratory distress syndrome (ARDS), ADAMTS-13 Deficiency, Aging choriocapillaris, aHUS, Allergic bronchitis bronchiectasis, Allergic bronchopulmonary aspergillosis (ABPA), allergy, Alzheimer's disease, AMD (wet and dry), Amyotrophic lateral sclerosis (ALS), And asbestos-induced inflammation, Anti-phospholipid syndrome (APLS), Arrhythmogenic Cardiomyopathy, Asthma, Atherosclerosis, Atypical hemolytic uremic syndrome (aHUS), Barraquer-Simons Syndrome, Behcet's disease, Berger's Disease/IgA nephropathy, Best disease (and pattern dystrophy), Bronchoconstriction, Bullous pemphigoid, C3 glomerulonephritis, Catastrophic anti-phospholipid syndrome (CAPS), Central retinal vein occlusion (CRVO), Cerebral Ischemia Reperfusion, Chagas Disease, Chorioretinal degenerations, Choroidal neovascularization (CNV), Chronic obstructive pulmonary disease (COPD), Cold agglutinin disease (CAD), cone degenerations, cone-rod dystrophies, Cranial nerve damage from meningitis, Creutzfeldt-jakob disease, Crohn's disease, Cystic fibrosis, Degenerative disc disease (DDD), Degos Disease, Dermatomyositis, Diabetic Nephropathy/Neuropathy, Diabetic retinal microangiopathy, Diabetic retinopathy macular edema, Diabetic retinopathy, Diseases presenting with thrombotic microangiopathy, Dominant drusen, dyspnea, hemoptysis, Emphysema, Endotoxemia, Eosinophilic pneumonia, endotheliopathy syndrome, Extracorporeal Circulation Disorders, pulmonary fibrosis and fibrotic disease, fibrogenic dust diseases, organ fibrosis, Giant cell aneurysm (GCA), glomerulonephritis, Graft vs Host Disease, Goodpasture's disease, Guillain-barre syndrome, Hemodialysis induced inflammation, Hemolytic anemia, Henoch-Schonlein purpura nephritis, Histoplasmosis of the eye, Huntington's disease, Hyperacute allograft rejection, Hypersensitivity pneumonitis, Hypertension-induced cardiac damage, Hypertension-induced fibrotic remodeling, Idiopathic neuropathic pain, Idiopathic polyneuropathy, Immune complex-associated inflammation, Interstitial lung disease, Ischemia-reperfusion injuries, Ischemia-reperfusion injury, Kawasaki disease, Malattia leventinese, Membranoproliferative glomerulonephritis, Membranous glomerulonephritis, Mesangioproliferative glomerulonephritis, MPGN II, Mucopolysaccharidoses, Multifocal motor neuropathy (MMN), Multiple sclerosis, Myasthenia gravis, myocardial infarction, neurological disorders, North Carolina macular dystrophy, organic dust diseases, Osteoarthritis, Parkinson's disease, Paroxysmal nocturnal hemoglobinuria (PNH), Pediatric Dense Deposit Disease, pemphigus vulgaris, photoreceptor degenerations, Polymyalgia rheumatica (PMR), Post-cardiopulmonary bypass inflammation, Post-streptococcal glomerulonephritis (PSGN), psoriasis, pulmonary embolisms and infarcts, pulmonary fibrosis, pulmonary vasculitis, Purtscher's retinopathy, Reactive airway disease syndrome, Renal cortical necrosis (RCN), Renal reperfusion injury, Respiratory syncytial virus (RSV), Retinal damage, Retinal degenerations, Retinal detachment, Retinal neovascularization, Retinal pigment epithelium (RPE) deposits, Rheumatoid arthritis, RPE degenerations, Secondary injury due to inflammation following traumatic injury, Sepsis, Sorsby's fundus dystrophy, Sorsby's fundus dystrophy, Spinal cord injury, Stargardt's disease, stroke, Systemic juvenile rheumatoid arthritis, systemic sclerosis, systemic lupus erythematosus (SLE), Systemic lupus erythematosus (SLE), Takayasu's arteritis, thermal injury including burns or frostbite, Transplant Rejection, Traumatic brain injury, Uveitis, Vascular leakage syndrome, Vasculitis, Vogt-Koyanagi-Harada syndrome, and Wegener's granulomatosis.

Diseases where complement byproducts plays a role in disease pathology are further listed by categories. AP activation is inhibited by AAC3b and therefore we except that the complement-mediated diseases will also be benefited.

Extracorporeal circulation disorders: Post-cardiopulmonary bypass inflammation, post-operative pulmonary dysfunction, cardiopulmonary bypass, hemodialysis, leukopheresis, plasmapheresis, plateletpheresis, heparin-induced extracorporeal LDL precipitation (HELP), postperfusion syndrome, extracorporeal membrane oxygenation (ECMO), cardiopulmonary bypass (CPB), post-perfusion syndrome, systemic inflammatory response, and multiple organ failure.

Cardiovascular disorders: acute coronary syndromes, Kawaski disease (arteritis), Takayasu's arteritis, Henoch-Schonlein purpura nephritis, vascular leakage syndrome, percutaneous coronary intervention (PCI), myocardial infarction, ischemia-reperfusion injury following acute myocardial infarction, atherosclerosis, vasculitis, immune complex vasculitis, vasculitis associated with rheumatoid arthritis (also called malignant rheumatoid arthritis), systemic lupus erythematosus-associated vasculitis, sepsis, arteritis, aneurysm, cardiomyopathy, dilated cardiomyopathy, cardiac surgery, peripheral vascular conditions, renovascular conditions, cardiovascular conditions, cerebrovascular conditions, mesenteric/enteric vascular conditions, diabetic angiopathy, venous gas embolus (VGE), Wegener's granulomatosis, heparin-induced extracorporeal membrane oxygenation, and Behcet's syndrome.

Bone/Musculoskeletal diseases and disorders: arthritis, inflammatory arthritis, non-inflammatory arthritis, rheumatoid arthritis, juvenile rheumatoid arthritis, systemic juvenile rheumatoid arthritis, osteoarthritis, osteoporosis, systemic lupus erythematosus (SLE), Behcet's syndrome, and Sjogren's syndrome.

Transplantation diseases and disorders: transplant rejection, xenograft rejection, graft versus host disease, xenotransplantation of organs or grafts, allotransplantation of organs or grafts, and hyperacute rejection.

Eye/Ocular diseases and disorders: wet and dry age-related macular degeneration (AMD), choroidal neovascularization (CNV), retinal damage, diabetic retinopathy, diabetic retinal microangiopathy, histoplasmosis of the eye, uveitis, diabetic macular edema, diabetic retinopathy, diabetic retinal microangiopathy, pathological myopia, central retinal vein occlusion (CRVO), corneal neovascularization, retinal neovascularization, retinal pigment epithelium (RPE), histoplasmosis of the eye, and Purtscher's retinopathy.

Hemolytic/Blood diseases and disorders: sepsis, systemic inflammatory response syndrome” (SIRS), hemorrhagic shock, acute respiratory distress syndrome (ARDS), catastrophic anti-phospholipid syndrome (CAPS), cold agglutinin disease (CAD), autoimmune thrombotic thrombocytopenic purpura (TTP), endotoxemia, hemolytic uremic syndrome (HUS), atypical hemolytic uremic syndrome (aHUS), paroxysmal nocturnal hemoglobinuria (PNH), sepsis, septic shock, sickle cell anemia, hemolytic anemia, hypereosinophilic syndrome, and anti-phospholipid syndrome (APLS).

Respiratory/Pulmonary diseases and disorders: asthma, Wegener's granulomatosis, transfusion-related acute lung injury (TRALI), antiglomerular basement membrane disease (Goodpasture's disease), eosinophilic pneumonia, hypersensitivity pneumonia, allergic bronchitis bronchiecstasis, reactive airway disease syndrome, respiratory syncytial virus (RSV) infection, parainfluenza virus infection, rhinovirus infection, adenovirus infection, allergic bronchopulmonary aspergillosis (ABPA), tuberculosis, parasitic lung disease, adult respiratory distress syndrome, chronic obstructive pulmonary disease (COPD), sarcoidosis, emphysema, bronchitis, cystic fibrosis, interstitial lung disease, acute respiratory distress syndrome (ARDS), transfusion-related acute lung injury, ischemia/reperfusion acute lung injury, byssinosis, heparin-induced extracorporeal membrane oxygenation, anaphylactic shock, and asbestos-induced inflammation.

Central and Peripheral Nervous System/Neurological diseases and disorders: multiple sclerosis (MS), myasthenia gravis (MG), myasthenia gravis, multiple sclerosis, Guillain Barre syndrome, Miller-Fisher syndrome, stroke, reperfusion following stroke, Alzheimer's disease, multifocal motor neuropathy (MMN), demyelination, Huntington's disease, amyotrophic lateral sclerosis (ALS), Parkinson's disease, degenerative disc disease (DDD), meningitis, cranial nerve damage from meningitis, variant Creutzfeldt-Jakob Disease (vCJD), idiopathic polyneuropathy, brain/cerebral trauma (including, but not limited to, hemorrhage, inflammation, and edema), and neuropathic pain.

Trauma-induced injuries and disorders: hemorrhagic shock, hypovolemic shock, spinal cord injury, neuronal injury, cerebral trauma, cerebral ischemia reperfusion, crush injury, wound healing, severe burns, and frostbite.

Renal diseases and disorders: renal reperfusion injury, poststreptococcal glomerulonephritis (PSGN), Goodpasture's disease, membranous nephritis, Berger's Disease/IgA nephropathy, mesangioproliferative glomerulonephritis, membranous glomerulonephritis, membranoproliferative glomerulonephritis (mesangiocapillary glomerulonephritis), acute postinfectious glomerulonephritis, cryoglobulinemic glomerulonephritis, lupus nephritis, Henoch-Schonlein purpura nephritis, and renal cortical necrosis (RCN).

Skin/Dermatologic diseases and disorders: burn injuries, psoriasis, atopic dermatitis (AD), eosinophilic spongiosis, urticaria, thermal injuries, pemphigoid, epidermolysis bullosa acquisita, autoimmune bullous dermatoses, bullous pemphigoid, scleroderma, angioedema, hereditary angioneurotic edema (HAE), erythema multiforme, herpes gestationis, Sjogren's syndrome, dermatomyositis, and dermatitis herpetiformis.

Gastrointestinal diseases and disorders: Crohn's disease, Celiac Disease/gluten-sensitive enteropathy, Whipple's disease, intestinal ischemia, inflammatory bowel disease, and ulcerative colitis.

Endocrine diseases and disorders: Hashimoto's thyroiditis, juvenile lymphocytic thyroiditis, stress anxiety, and other diseases affecting prolactin, growth or insulin-like growth factor, adrenocorticotropin release, pancreatitis, Addison's disease, diabetic conditions including, but not limited to, type 1 and type 2 diabetes, type I diabetes mellitus, sarcoidosis, diabetic retinal microangiopathy, non-obese diabetes (IDDM), angiopathy, neuropathy or retinopathy complications of IDDM or Type-2 diabetes, and insulin resistance.

Reperfusion injuries and disorders of organs: including but not limited to heart, brain, kidney, and liver.

Reproduction and urogenital diseases and disorders: painful bladder diseases and disorders, sensory bladder diseases and disorders, spontaneous abortion, male and female diseases from infertility, diseases from pregnancy, fetomaternal tolerance, pre-eclampsia, urogenital inflammatory diseases, diseases and disorders from placental dysfunction, diseases and disorders from miscarriage, chronic bacterial cystitis, and interstitial cystitis.

### EXAMPLES

Unless stated otherwise, all reagents were of high grade available. All complement proteins, alternative and classical pathway buffers, detection antibodies, and erythrocytes were from Complement Technologies (Tyler, Tex.) or Quidel Corporation (San Diego, Calif.). All secondary antibodies were from American Qualex, San Clemente, Calif., BSA and other reagents were all from Sigma-Aldrich, St Louis, Mo.

### Example 1

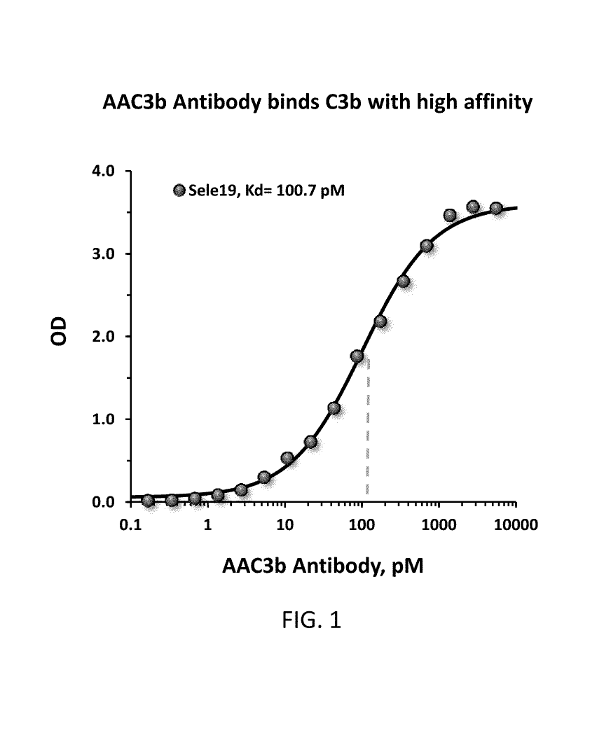

### Humanized AAC3b Binds C3b with High Affinity (FIG. 1)

Methods—To perform this experiment, polystyrene microtiter plates were coated with human C3b (2.0 μg/50 μl per well) in phosphate buffered saline (PBS) overnight at 4° C. After aspirating the C3b solution, the wells were blocked with PBS containing 1% bovine serum albumin (BSA) (Sigma-Aldrich, St. Louis, Mo.) for 1 hour at room temperature. Wells without C3b coating served as background controls. Aliquots of AAC3b were added to C3b coated wells and allowed to incubate for 1 hour to allow binding. Following this incubation at room temperature, the plate was rinsed with PBS and incubated with 1:2000 diluted peroxidase-conjugated goat anti-human monoclonal antibody. Following this incubation, the plate was rinsed and the bound peroxidase was identified using TMB reagent. TMB solution (KPL, Gaithersburg, Md.) was then added and allowed to incubate for 30 min at room temperature. TMB Stop solution (KPL, Gaithersburg, Md.) was then added to all plate wells. Immediately following addition of stop solution, the plate(s) were read in a microplate reader at 450 nm. As shown in FIG. 1, AAC3b binds C3b with 100 pM affinity.

### Example 2

### AAC3b Antibody Inhibits Alternative Pathway (AP) Dependent Lysis of Rabbit Red Blood Cell (rRBC) in Minimally Diluted Normal Human Serum (NHS)

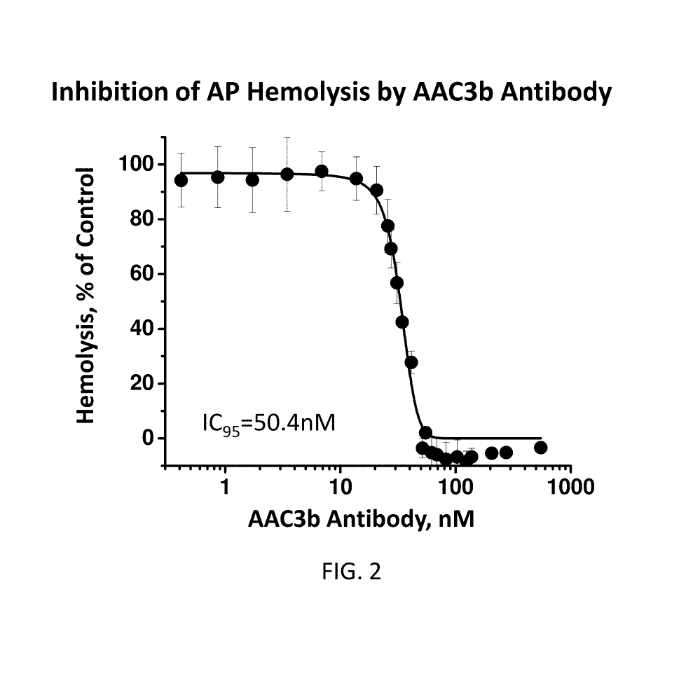

This erythrocyte lysis assay is based on the formation of terminal complement complex on the surface of the rRBC (rabbit Red Blood Cell) in Normal Human Serum. As a result, the rRBCs are lysed. The progressive decrease in light scatter at 700 nm is a direct measure of erythrocyte lysis. Typically, rRBC(s) are incubated in normal human serum in gelatin veronal buffer containing 10 mM MgCl2/EGTA. Under these conditions, the surface of rRBC triggers the activation of alternative pathway in normal human serum. The alternative pathway activation leads to the formation of C5b-9 complex on the surface of the rRBC(s). Agents that inhibit the formation of C5b-9 complexes are expected to inhibit cellular lysis. To evaluate the effect of AAC3b antibody, the antibody was incubated with normal human serum (90% NHS) in AP buffer at 37° C. with a fixed concentration of rabbit erythrocytes in a temperature controlled ELISA plate reader capable of reading at 700 nm. A progressive decrease in light scatter (due to lysis of intact cells) was measured at 700 nm as a function of time. The data were recorded and analyzed with a SpectraMax 190 plate reader and SoftMax software. For calculation total inhibition was calculated to be at 50 nM in 90% NHS. As shown in FIG. 2, AAC3b inhibits AP mediated hemolysis at 50 nM in 90% normal human serum. The antibody binds C3b and not C3.

### Example 3

### AAC3b Antibody Inhibits Alternative Pathway (AP) Dependent Lysis of Rabbit Red Blood Cell (rRBC). Classical Pathway Dependent Lysis of Antibody Sensitized Sheep Erythrocytes in Normal Human Serum is not Inhibited

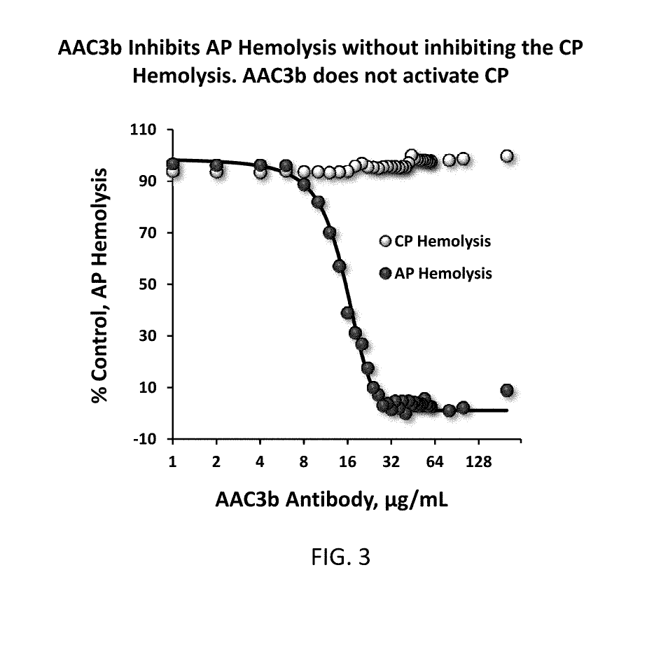

The AP hemolysis assay was conducted as described for Example 2. For the CP lysis assay which was conducted in 2% and 20% normal human serum. Antibody sensitized sheep erythrocytes were incubated with 2% or 20% NHS and data was recorded at OD700 nm as a function of time. The data were recorded and analyzed with a SpectraMax 190 plate reader and SoftMax software. AAC3b did not inhibit classical pathway activation in CP buffer. Thus, AAC3b is a selective inhibitor AP activation but not CP as shown in FIG. 3.

### Example 4

### AAC3b does not Bind C1Q in Normal Human Serum

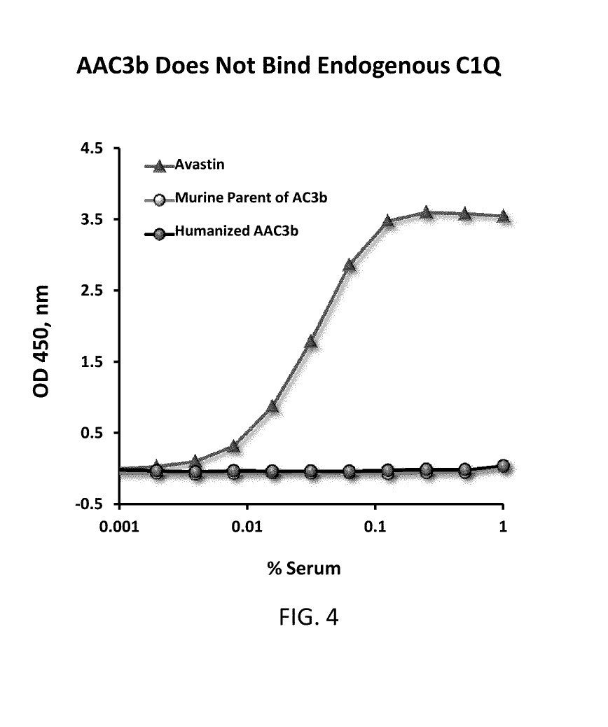

C1Q (present in Normal Human Serum) does not bind the substrate-bound AAC3b antibody as shown in FIG. 4. ELISA wells were coated with AAC3b and Avastin and incubated overnight at 4° C. Following incubation, the coating solutions were aspirated and the wells were blocked with 1% BSA in PBS. C1Q is present in serum and therefore was used as a source for C1Q for the assay. Normal human serum at 1% concentration was added to both Avastin and AAC3b coated wells. Following incubation at 37° C. for two hours, the plate was washed and the bound C1Q was detected with a 1:2000 dilution of Goat Anti-C1q primary antibody. A Rabbit Anti-Goat HRP was used as the secondary antibody for detection. Following one hour incubation at room temperature, HRP color was developed with TMB solution, which was allowed to incubate for 30 min at room temperature. TMB Stop solution was then added to all wells. The plates were read immediately after addition of stop solution in a microplate reader at 450 nm. As shown in FIG. 4, AAC3b does not bind C1Q, unlike Avastin, which displayed maximum binding saturation.

### Example 5

### ELISA for Detection of Convertase Formation on LPS

Alternative complement pathway is activated in normal human serum by lipopolysaccharide (LPS). We used this assay to demonstrate whether AAC3b antibody would inhibit the formation of C3 and C5 convertases. Properdin, C3b, and Bb are the components of the C3 and C5 convertases. Additionally C5b-9 formation represents the final terminal complement complex (TCC). We therefore measured the deposition of P, C3b, Bb, and C5b-9 in the presence and absence of the AAC3b antibody. The deposited P, C3b, Bb, and C5b-9 were detected with appropriate antibodies. In the presence of AAC3b antibody, a dose dependent inhibition of C3 and C5 convertase formation was noticed as indicated by the inhibition of deposition of each of the P, C3b, Bb, and C5b-9 molecules. AP C3 Convertase and AP C5 convertase were associated with cell membrane in vivo. In an in vitro assay, these convertases deposit onto LPS coated assay wells. Similarly, AP activation results in the formation and deposition of C5b-9. An ELISA method was used to evaluate the effect of AAC3b antibody on the formation and deposition of AP C3 Convertase, AP C5 convertase, and MAC.

LPS (4 μg/100 μL) was added to ELISA. Coated wells were blocked with 1% BSA in PBS. A solution of 10% normal human serum in AP buffer (GVB, 10 mM Mg EGTA, pH 7.3) was used as a negative control for total AP activation. To test the effect of AAC3b antibody, various concentrations of this antibody were mixed with 10% NHS. NHS with and without AAC3b was incubated on LPS coated plates at 37° C. for 2 hours at RT to allow AP activation to occur. Deposited complement components were detected with appropriate antibodies; anti-properdin antibody detected the deposited properdin, anti-C3c antibody detected the deposited C3b and anti-Factor B detected the deposited Bb, and anti-C5b-9 detected the MAC/TCC. Following incubation with the peroxidase conjugated secondary antibody, plates were developed with TMB solution and the color development proceeded for 30 min at room temperature. TMB Stop solution was then added to all wells and the plates were read at 450 nm.

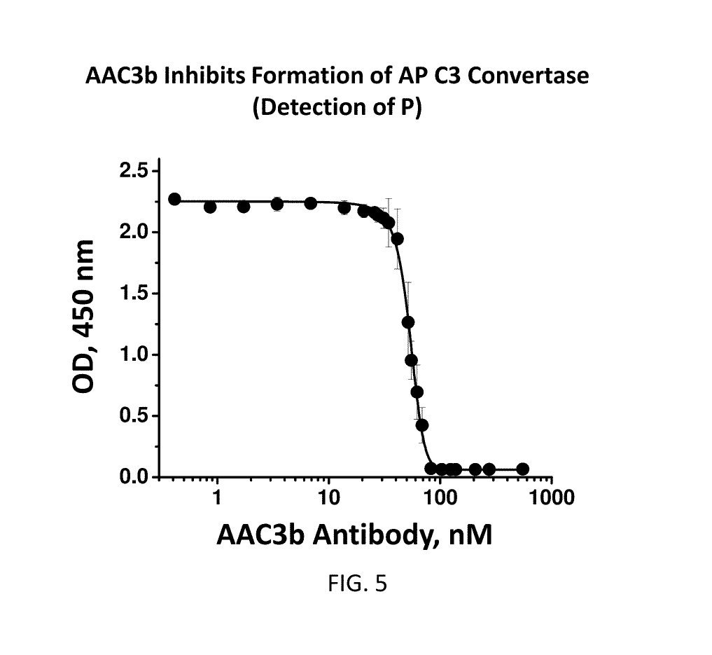

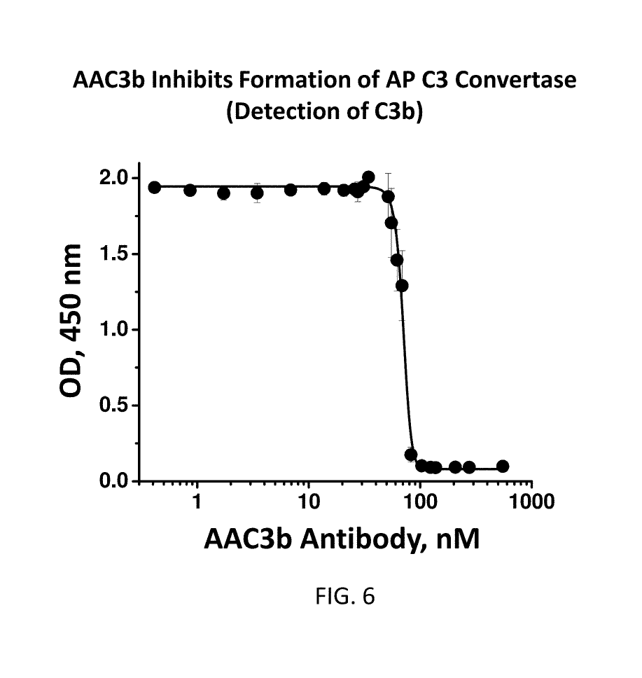

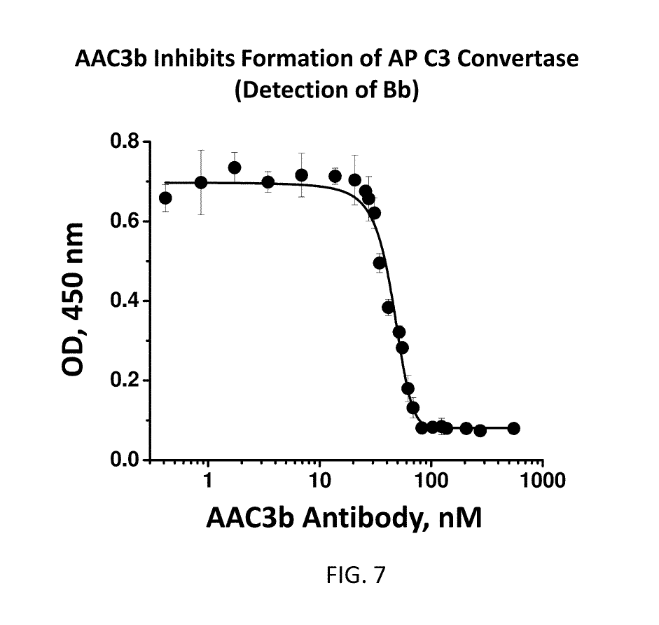

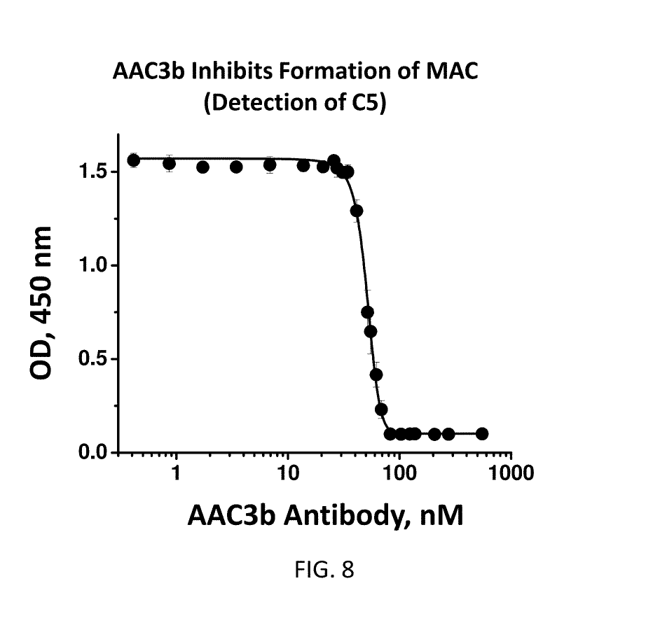

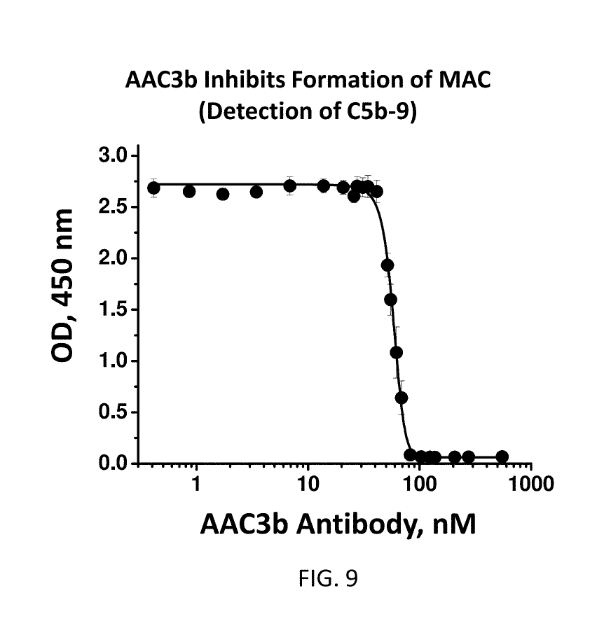

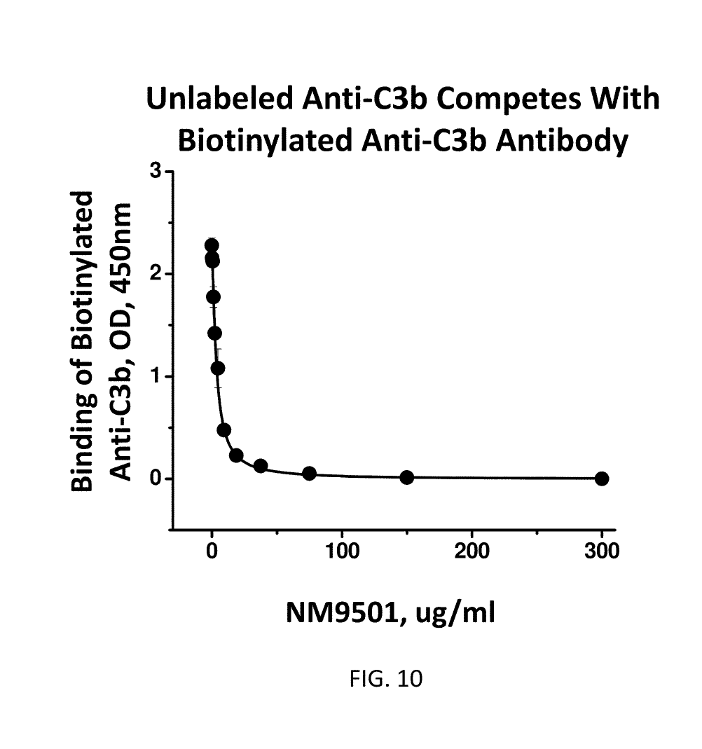

FIGS. 5, 6, 7, 8, 9 demonstrate dose-dependent inhibition of properdin formation and deposition, C3b formation and deposition, Bb formation and deposition, and C5b-9 formation and deposition. These data demonstrate that AAC3b antibody inhibits the formation of both AP C3 and AP C5 convertases in 10% NHS. FIG. 10 shows data on MAC inhibition. These results demonstrate that the AAC3b antibody is capable to preventing the formation and deposition of C3/C5 convertases and MAC formation and deposition.

### Example 6

### Antibodies that Compete with AAC3b Antibody

Antibodies with similar CDRs sequences are expected to bind to the same epitope on a target antigen. Minor changes in the amino acid sequences of the CDRs may reduce the binding affinity of the antibody to the target antigen but still compete with the antibody for binding. Depending upon the situation, one may see 100% competition or as low as 50% competition. Antibodies that compete with the AAC3b antibody exhibit similar pharmacological effects in vivo.

ELISA wells were coated with 1 μg/100 μL per well of the C3b. Plates were incubated with the coating solution in cold at 4° C. over night. The coating solutions was aspirated and wells were treated with 1% BSA in PBS for 2 hours at room temperature. Biotinylated AAC3b antibody at fixed concentration was mixed with various concentrations of unlabeled AAC3b and this solution was aliquoted into the C3b coated wells. Following a 2 hour incubation at RT, the plate was rinsed with PBS and incubated with 1:2000 diluted peroxidase-conjugated neutravidin. Following this incubation, the plate was rinsed and the bound peroxidase was identified using TMB reagent. TMB solution (KPL, Gaithersburg, Md.) was then added and allowed to incubate for 30 min at room temperature. TMB Stop solution (KPL, Gaithersburg, Md.) was then added to all plate wells. Immediately following addition of stop solution, the plate(s) were read in a microplate reader at 450 nm. As shown in FIG. 10, unlabeled AAC3b antibody competes with Biotinylated AAC3b antibody suggesting that both antibodies share the same epitope on C3b.

### Example 7

### Aglycosylated Fc does not Bind to CD16a, CD16b or CD32a/b/c

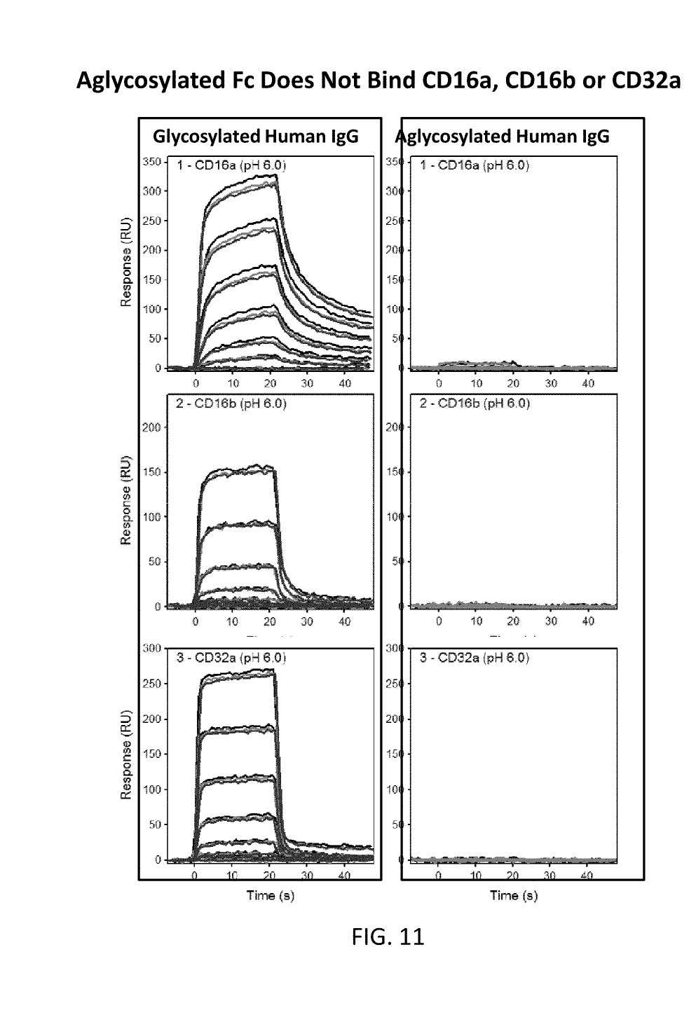

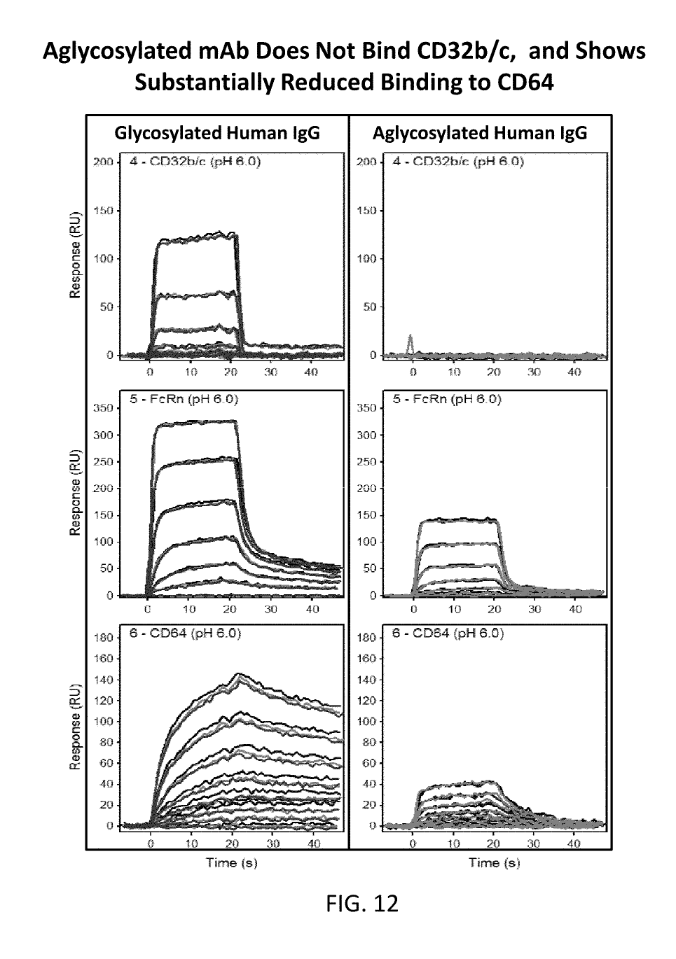

BIACORE methods were used for evaluating this binding. Appropriate Fc receptors were coated and the binding was evaluated. As shown in FIGS. 11 and 12, aglycosylated Fc has low to no binding to Fc receptors except FcRn.

### Example 8

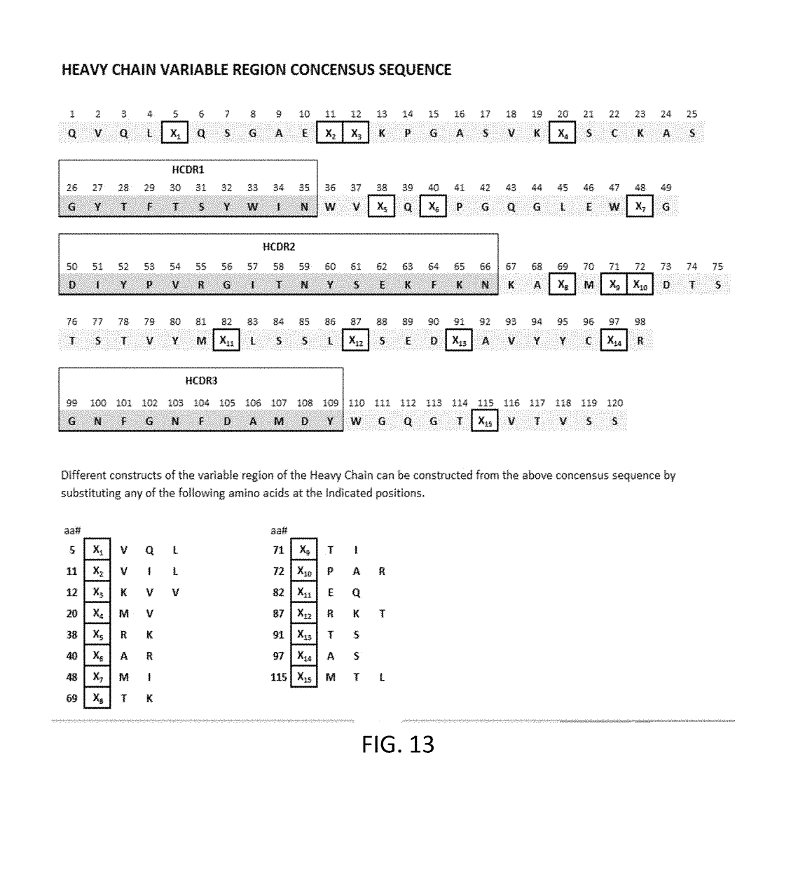

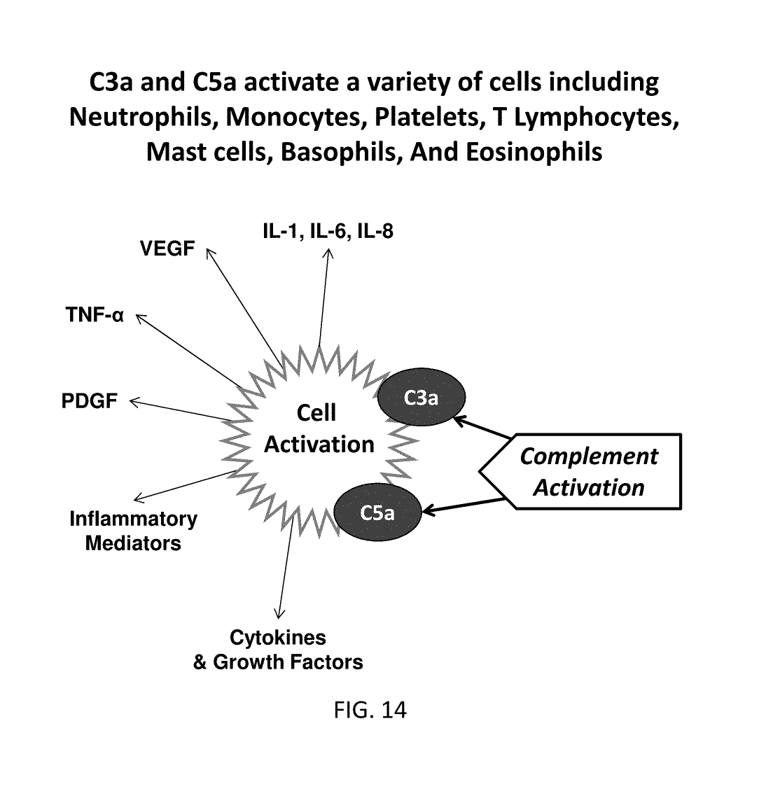

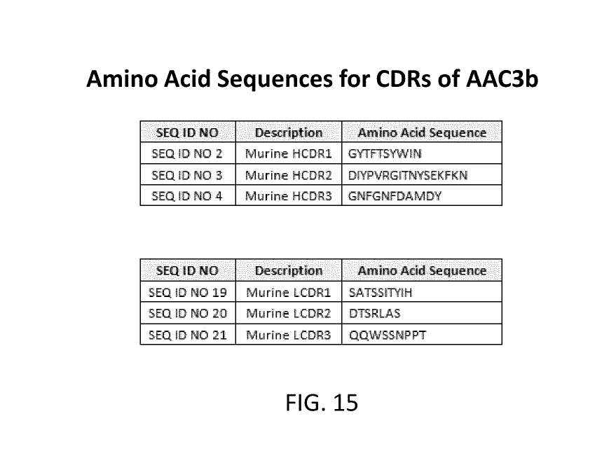

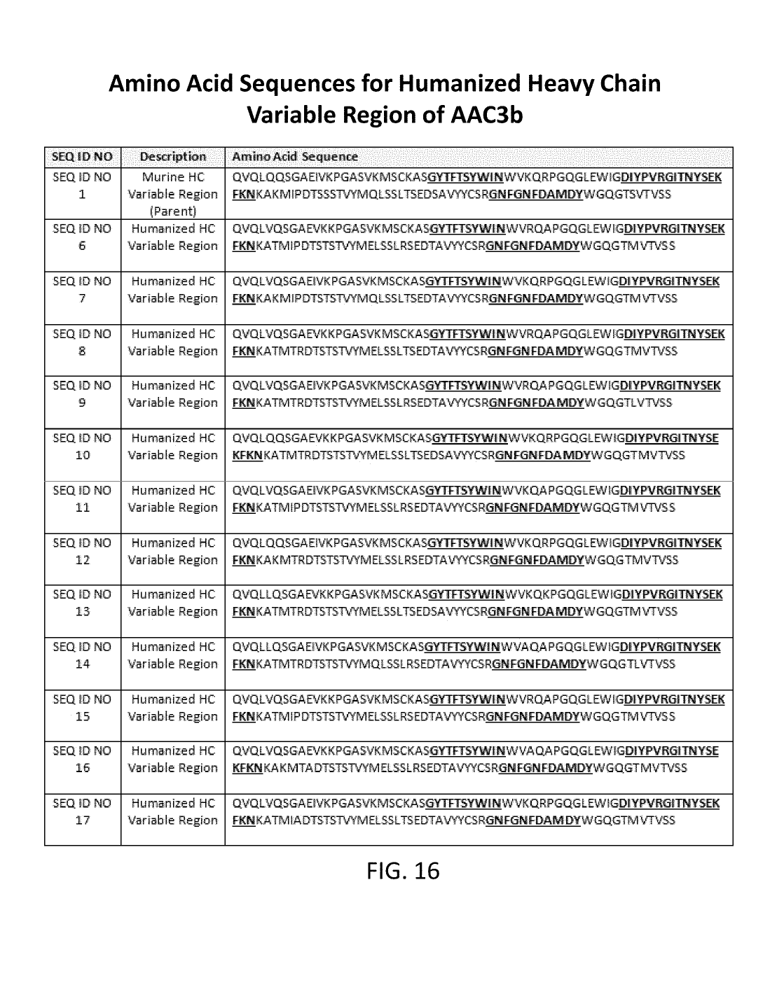

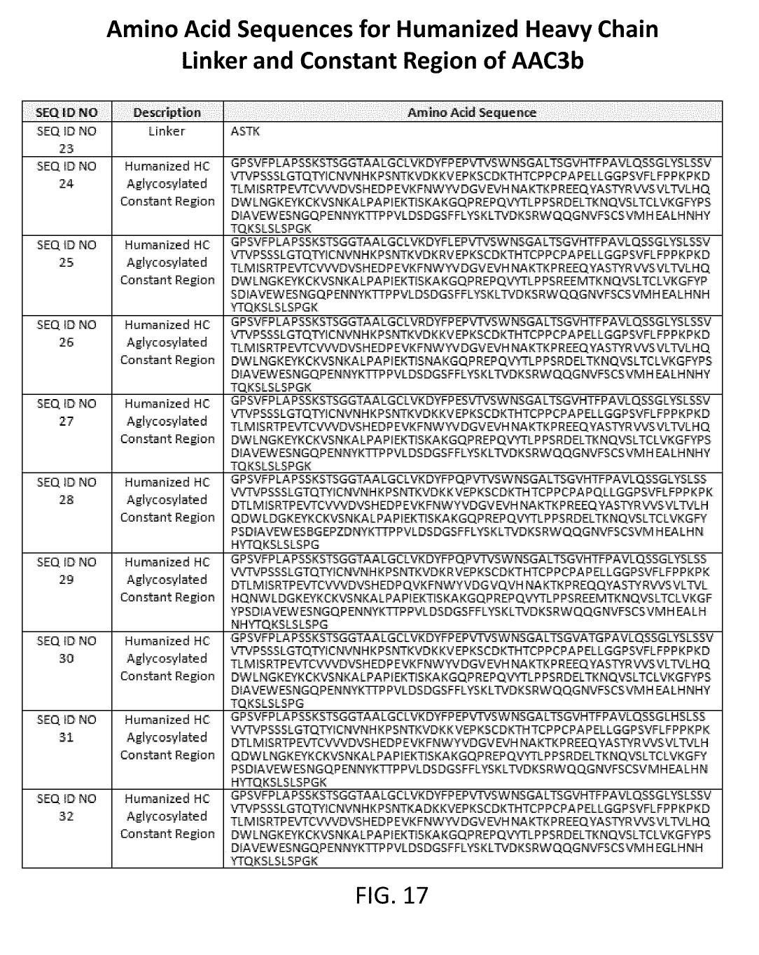

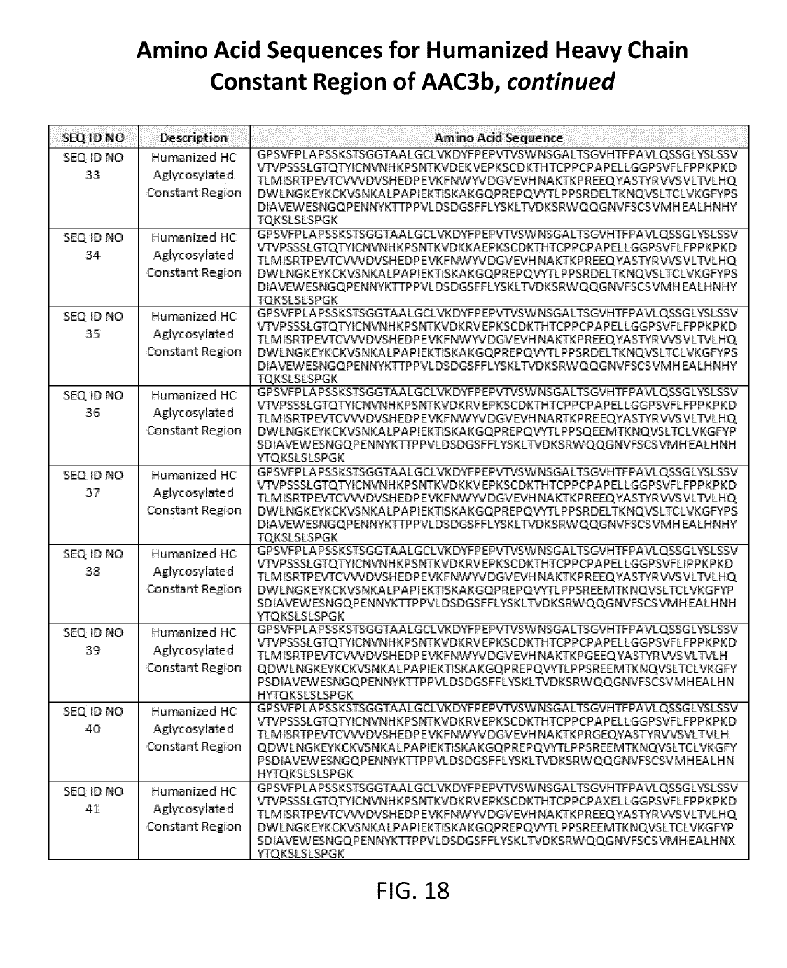

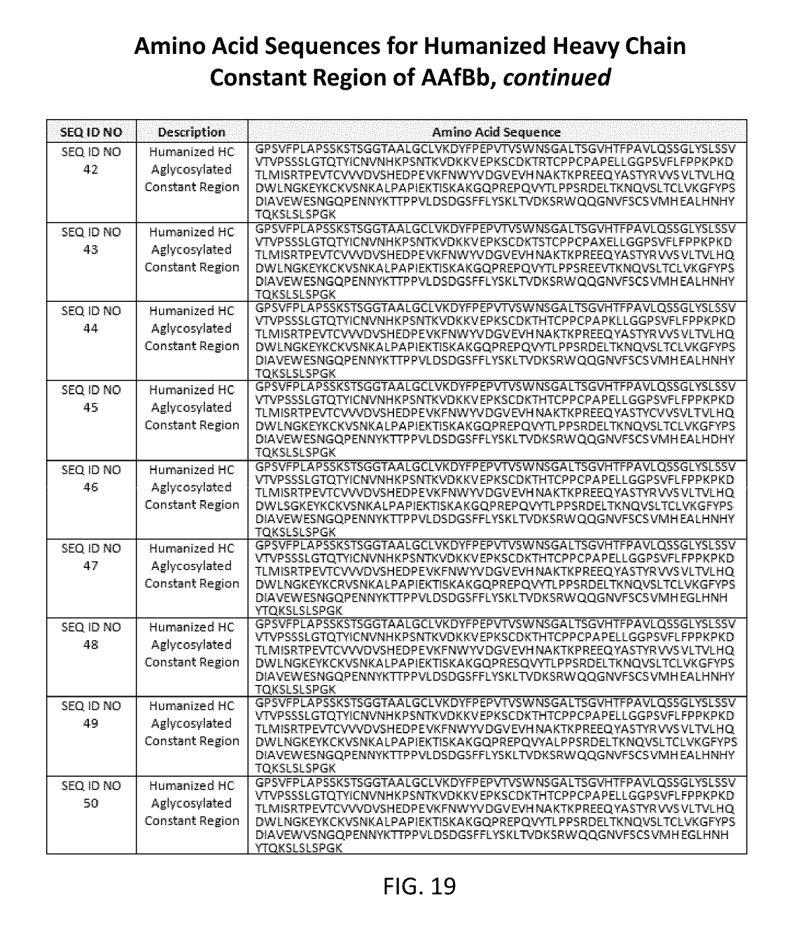

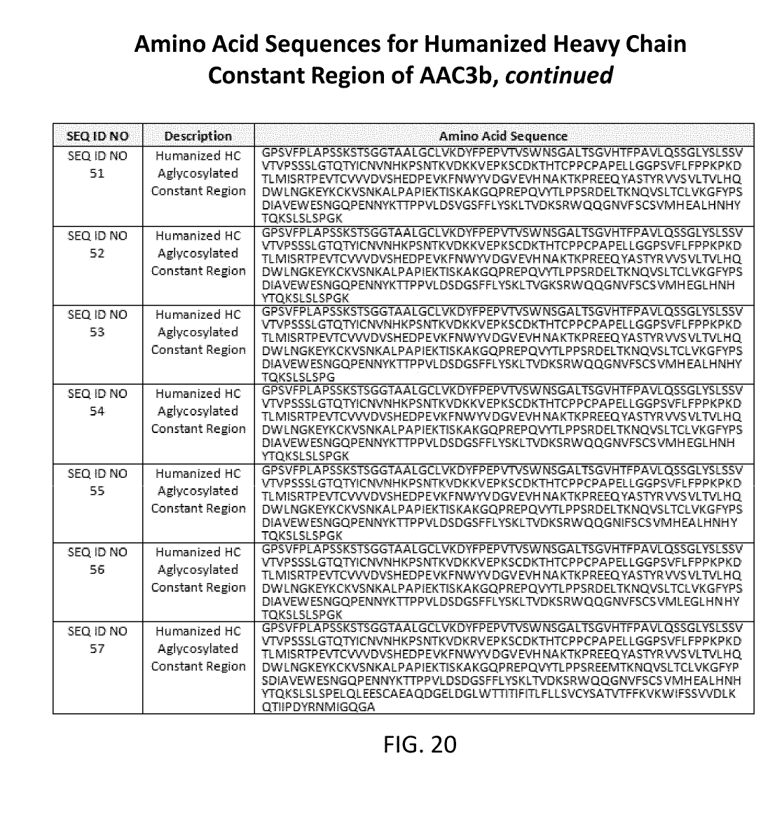

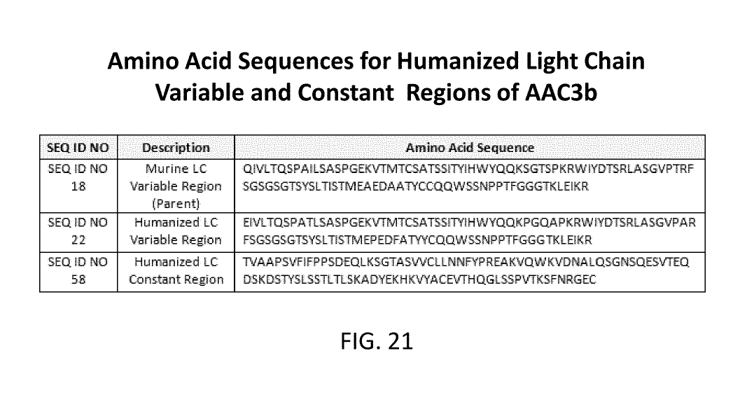

Heavy chain profile from multiple aglycosylated AAC3b is shown. An antibody can be constructed from any Heavy chain variable region linked to any heavy chain constant region as shown in FIGS. 13, 15, 16, 17, 18, 19, 20 and 21.

### Example 9

### Production of Humanized Anti-Bb Antibodies

A murine monoclonal antibody harboring the CDRs was sequenced and the CDRs were grafted in human framework regions. Following codon optimization, the sequences were expressed in CHO cells for the production of AAC3b antibodies. Aglycosylated antibodies and its fragments can be expressed in any type of CHO cells or any cell that can express mammalian antibodies.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. An aglycosylated humanized anti-C3b (AAC3b) antibody or antigen binding fragment thereof, comprising a modification at a conserved N-linked site in the CH2 domains of an Fc portion of the antibody or antigen binding fragment thereof.

2. The AAC3b antibody or antigen binding fragment thereof of claim 1, wherein the modification comprises a mutation in the heavy chain glycosylation site, wherein the mutation prevents glycosylation at the site.

3. The AAC3b antibody or antigen binding fragment thereof of claim 2, wherein the modification comprises a mutation of N298Q (N297 using EU Kabat numbering).

4. The AAC3b antibody or antigen binding fragment thereof of claim 2, wherein the modification comprises a mutation of N298A (N297 using EU Kabat numbering).

5. The AAC3b antibody or antigen binding fragment thereof of claim 1, wherein the modification comprises the removal of the CH2 domain glycans.

6. The AAC3b antibody or antigen binding fragment thereof of claim 1, wherein the modification prevents glycosylation at the CH2 domain.

7. The AAC3b antibody or antigen binding fragment thereof of claim 1, wherein the AAC3b antibody or antigen binding fragment thereof does not bind to an Fc effector receptor and/or does not cause cellular lysis.

9. The AAC3b antibody or antigen binding fragment thereof of claim 1, wherein the antibody is selected from the group consisting of: monoclonal antibodies, polyclonal antibodies, murine antibodies, chimeric antibodies, primatized antibodies, and humanized antibodies.

10. The AAC3b antibody or antigen binding fragment thereof of claim 1, wherein the antibody is selected from the group consisting of: multimeric antibodies, heterodimeric antibodies, hemidimeric antibodies, tetravalent antibodies, bispecific antibodies, Fab, Fab′, Fab′2, F(v) antibody fragments, and single chain antibodies or derivatives thereof.

11. The AAC3b antibody or antigen binding fragment thereof of claim 1, including a humanized heavy chain aglycosylated region having an amino acid sequence selected from the group consisting of: SEQ ID NOs: 24-56, and 57.

12. The AAC3b antibody or antigen binding fragment thereof of claim 1, being an aglycosylated humanized antibody or antigen binding fragment thereof of a murine monoclonal antibody produced by a hybridoma cell deposited under ATCC Accession No. PTA-8806.

13. The AAC3b antibody or antigen binding fragment thereof of claim 1, having a heavy chain variable domain including 3CDRs having the amino acid sequences of SEQ ID NO: 2, SEQ ID NO: 3 and SEQ ID NO: 4 and a light chain variable domain including 3CDRs having amino acid sequences of SEQ ID NO: 19, SEQ ID NO: 20, and SEQ ID NO: 21

14. The AAC3b antibody or antigen binding fragment thereof of claim 1, having a heavy chain variable domain with an amino acid sequence at least 90% identical to SEQ ID NO: 1.

15. The AAC3b antibody or antibody derivative of claim 1, being conjugated to a detectable marker, therapeutic agent, imaging agent, or radionuclide.

16. A method for inhibiting alternative complement pathway in a subject in need thereof, comprising: administering to the subject a therapeutically effective amount of an aglycosylated humanized anti-C3b (AAC3b) antibody or antigen binding fragment thereof, wherein the AAC3b antibody or antigen binding fragment thereof has similar affinity binding to C3b as a murine anti-C3b antibody produced by the hybridoma cell line deposited under ATCC Accession No. PTA-8806.

17. The method according to claim 16, wherein the AAC3b antibody or antigen binding fragment thereof having at least one of the following properties: specifically binds C3b and prevents formation of C3a and C3b; specifically binds C3b and prevents formation of C5a and C5b, specifically binds C3b and prevents formation of SC5b-9, C5b-6, C5b-7, C5b-8, and C5b-9, specifically binds C3b and prevents formation and deposition of C3b, specifically binds C3b and prevents formation and deposition of PC3b, specifically binds C3b and prevents formation and deposition of PC3bBb, specifically binds C3b and prevents formation and deposition of (P)n(C3b)n(Bb)n where n is equal to any value between 1 to 10, specifically binds C3b and prevents activation of neutrophils, monocytes, and platelets via the inhibition of AP, specifically binds C3b and prevents formation of various cytokines including VEGF and IL-1, specifically binds C3b and prevents lysis of erythrocytes that do lack or do not carry human CD55 or CD59, or specifically binds C3b and prevents lysis of platelets.

18. A method of ameliorating complement-mediated diseases in a subject in need thereof, the method comprising:
administering to the subject a therapeutically effective amount of an aglycosylated humanized anti-C3b (AAC3b) antibody or antigen binding fragment thereof, wherein the antibody or antigen binding fragment thereof includes a mutation of N297 using EU Kabat numbering at the conserved N-linked sites in the CH2 domains of an Fc portion of the AAC3b antibody or antigen binding fragment thereof, wherein the mutation prevents glycosylation at the site and binding to Fc receptors on cells.

19. The method of claim 18, wherein the AAC3b antibody or antigen binding fragment thereof has similar affinity binding to C3b as a murine anti-C3b antibody produced by the hybridoma cell line deposited under ATCC Accession No. PTA-8806.

20. The method of claim 18, wherein the complement mediated disease are selected from the group consisting of inflammatory disorders, Extracorporeal Circulation Disorders, Cardiovascular Disorders, Musculoskeletal Disorders, Ocular Disorders, Transplantation disease Disorders, Hemolytic Disorders, Repiratory Disorders, Neurological Disorders, Trauma-induced Disorders, Renal Disorders, Dematological Disorders, Gastrointestinal Disorders, Endocrine Disorders, Reproduction and urogenital diseases and disorders, and Reperfusion Injury Disorders.


SEQUENCE LISTING (58 sequences)

SEQ ID NO: 1 - protein, 120 aa, Mus musculus
QVQLQQSGAEIVKPGASVKMSCKASGYTFTSYWINWVKQRPGQGLEWIGDIYPVRGITNYSEKFKNKAKMIPDTSSSTVYMQLSSLTSEDSAVYYCSRGNFGNFDAMDYWGQGTSVTVSS

SEQ ID NO: 2 - protein, 10 aa, Mus musculus
GYTFTSYWIN

SEQ ID NO: 3 - protein, 17 aa, Mus musculus
DIYPVRGITNYSEKFKN

SEQ ID NO: 4 - protein, 11 aa, Mus musculus
GNFGNFDAMDY

SEQ ID NO: 5 - protein, 120 aa, Artificial Sequence
  Synthetic Construct
  MISC_FEATURE (5)..(5): Xaa is any naturally occuring amino acid
  MISC_FEATURE (11)..(12): Xaa is any naturally occuring amino acid
  MISC_FEATURE (20)..(20): Xaa is any naturally occuring amino acid
  MISC_FEATURE (38)..(38): Xaa is any naturally occuring amino acid
  MISC_FEATURE (40)..(40): Xaa is any naturally occuring amino acid
  MISC_FEATURE (48)..(48): Xaa is any naturally occuring amino acid
  MISC_FEATURE (69)..(69): Xaa is any naturally occuring amino acid
  MISC_FEATURE (71)..(72): Xaa is any naturally occuring amino acid
  MISC_FEATURE (82)..(82): Xaa is any naturally occuring amino acid
  MISC_FEATURE (87)..(87): Xaa is any naturally occuring amino acid
  MISC_FEATURE (91)..(91): Xaa is any naturally occuring amino acid
  MISC_FEATURE (97)..(97): Xaa is any naturally occuring amino acid
  MISC_FEATURE (115)..(115): Xaa is any naturally occuring amino acid
QVQLXQSGAEXXKPGASVKXSCKASGYTFTSYWINWVXQXPGQGLEWXGDIYPVRGITNYSEKFKNKAXMXXDTSTSTVYMXLSSLXSEDXAVYYCXRGNFGNFDAMDYWGQGTXVTVSS

SEQ ID NO: 6 - protein, 120 aa, Artificial Sequence
  Synthetic Construct
QVQLVQSGAEVKKPGASVKMSCKASGYTFTSYWINWVRQAPGQGLEWIGDIYPVRGITNYSEKFKNKATMIPDTSTSTVYMELSSLRSEDTAVYYCSRGNFGNFDAMDYWGQGTMVTVSS

SEQ ID NO: 7 - protein, 120 aa, Artificial Sequence
  Synthetic Construct
QVQLVQSGAEIVKPGASVKMSCKASGYTFTSYWINWVKQRPGQGLEWIGDIYPVRGITNYSEKFKNKAKMIPDTSTSTVYMQLSSLTSEDTAVYYCSRGNFGNFDAMDYWGQGTMVTVSS

SEQ ID NO: 8 - protein, 120 aa, Artificial Sequence
  Synthetic Construct
QVQLVQSGAEVKKPGASVKMSCKASGYTFTSYWINWVRQAPGQGLEWIGDIYPVRGITNYSEKFKNKATMTRDTSTSTVYMELSSLTSEDTAVYYCSRGNFGNFDAMDYWGQGTMVTVSS

SEQ ID NO: 9 - protein, 120 aa, Artificial Sequence
  Synthetic Construct
QVQLVQSGAEIVKPGASVKMSCKASGYTFTSYWINWVRQAPGQGLEWIGDIYPVRGITNYSEKFKNKATMTRDTSTSTVYMELSSLRSEDTAVYYCSRGNFGNFDAMDYWGQGTLVTVSS

SEQ ID NO: 10 - protein, 120 aa, Artificial Sequence
  Synthetic Construct
QVQLQQSGAEVKKPGASVKMSCKASGYTFTSYWINWVKQRPGQGLEWIGDIYPVRGITNYSEKFKNKATMTRDTSTSTVYMELSSLTSEDSAVYYCSRGNFGNFDAMDYWGQGTMVTVSS

SEQ ID NO: 11 - protein, 120 aa, Artificial Sequence
  Synthetic Construct
QVQLVQSGAEIVKPGASVKMSCKASGYTFTSYWINWVKQAPGQGLEWIGDIYPVRGITNYSEKFKNKATMIPDTSTSTVYMELSSLRSEDTAVYYCSRGNFGNFDAMDYWGQGTMVTVSS

SEQ ID NO: 12 - protein, 120 aa, Artificial Sequence
  Synthetic Construct
QVQLQQSGAEIVKPGASVKMSCKASGYTFTSYWINWVKQRPGQGLEWIGDIYPVRGITNYSEKFKNKAKMTRDTSTSTVYMELSSLRSEDTAVYYCSRGNFGNFDAMDYWGQGTMVTVSS

SEQ ID NO: 13 - protein, 120 aa, Artificial Sequence
  Synthetic Construct
QVQLLQSGAEVKKPGASVKMSCKASGYTFTSYWINWVKQKPGQGLEWIGDIYPVRGITNYSEKFKNKATMTRDTSTSTVYMELSSLTSEDSAVYYCSRGNFGNFDAMDYWGQGTMVTVSS

SEQ ID NO: 14 - protein, 120 aa, Artificial Sequence
  Synthetic Construct
QVQLLQSGAEIVKPGASVKMSCKASGYTFTSYWINWVAQAPGQGLEWIGDIYPVRGITNYSEKFKNKATMTRDTSTSTVYMQLSSLRSEDTAVYYCSRGNFGNFDAMDYWGQGTLVTVSS

SEQ ID NO: 15 - protein, 120 aa, Artificial Sequence; identical to SEQ ID NO: 6
  Synthetic Construct

SEQ ID NO: 16 - protein, 120 aa, Artificial Sequence
  Synthetic Construct
QVQLVQSGAEVKKPGASVKMSCKASGYTFTSYWINWVAQAPGQGLEWIGDIYPVRGITNYSEKFKNKAKMTADTSTSTVYMELSSLRSEDTAVYYCSRGNFGNFDAMDYWGQGTMVTVSS

SEQ ID NO: 17 - protein, 120 aa, Artificial Sequence
  Synthetic Construct
QVQLVQSGAEIVKPGASVKMSCKASGYTFTSYWINWVKQRPGQGLEWIGDIYPVRGITNYSEKFKNKATMIADTSTSTVYMELSSLTSEDTAVYYCSRGNFGNFDAMDYWGQGTMVTVSS

SEQ ID NO: 18 - protein, 107 aa, Mus musculus
QIVLTQSPAILSASPGEKVTMTCSATSSITYIHWYQQKSGTSPKRWIYDTSRLASGVPTRFSGSGSGTSYSLTISTMEAEDAATYCCQQWSSNPPTFGGGTKLEIKR

SEQ ID NO: 19 - protein, 10 aa, Mus musculus
SATSSITYIH

SEQ ID NO: 20 - protein, 7 aa, Mus musculus
DTSRLAS

SEQ ID NO: 21 - protein, 9 aa, Mus musculus
QQWSSNPPT

SEQ ID NO: 22 - protein, 107 aa, Artificial Sequence
  Synthetic Construct
EIVLTQSPATLSASPGEKVTMTCSATSSITYIHWYQQKPGQAPKRWIYDTSRLASGVPARFSGSGSGTSYSLTISTMEPEDFATYYCQQWSSNPPTFGGGTKLEIKR

SEQ ID NO: 23 - protein, 4 aa, Artificial Sequence
  Synthetic Construct
ASTK

SEQ ID NO: 24 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 326 aa, Artificial Sequence
  Synthetic Construct
GPSVFPLAPSSKSTSGGTAALGCLVKDYFLEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 326 aa, Artificial Sequence
  Synthetic Construct
GPSVFPLAPSSKSTSGGTAALGCLVRDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISNAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 326 aa, Artificial Sequence
  Synthetic Construct
GPSVFPLAPSSKSTSGGTAALGCLVKDYFPESVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 325 aa, Artificial Sequence
  Synthetic Construct
GPSVFPLAPSSKSTSGGTAALGCLVKDYFPQPVTVSWNSGALTSGVHTFPAVLQSSGLYSLSSVVTVPSSSLGTQTYICNVNHKPSNTKVDKKVEPKSCDKTHTCPPCPAPQLLGGPSVFLFPPKPKDTLMISRTPEVTCVVVDVSHEDPEVKFNWYVDGVEVHNAKTKPREEQYASTYRVVSVLTVLHQDWLDGKEYKCKVSNKALPAPIEKTISKAKGQPREPQVYTLPPSRDELTKNQVSLTCLVKGFYPSDIAVEWESBGEPZDNYKTTPPVLDSDGSFFLYSKLTVDKSRWQQGNVFSCSVMHEALHNHYTQKSLSLSPG

SEQ ID NO: 29 - protein, 325 aa, Artificial Sequence
  Synthetic Construct
GPSVFPLAPSSKSTSGGTAALGCLVKDYFPQPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPQVKFNWYVDGVQVHNAKTKPREQQYASTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 325 aa, Artificial Sequence
  Synthetic Construct
GPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 326 aa, Artificial Sequence
  Synthetic Construct
GPSVFPLAPSSKSTSGGTAALGCLVKDYFPEPVTVSWNSGALTSGVHTFPAVLQSSGLHSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKADKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 33 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNARTKPREEQYASTYRVVSVLTVLHQDWLNGKEYKCKVSNKALPAPIEKTISKAKGQPREPQVYTLPPSQEEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLIPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPGEEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPRGEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 326 aa, Artificial Sequence
  Synthetic Construct
  MISC_FEATURE (111)..(111): Xaa is any naturally occurring  amino acid
  MISC_FEATURE (314)..(314): Xaa is any naturally occurring  amino acid
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAXELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNXYTQKSLSLSPGK

SEQ ID NO: 41 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTRTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 326 aa, Artificial Sequence
  Synthetic Construct
  MISC_FEATURE (111)..(111): Xaa is any naturally occurring amino acid
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSTCPPCPAXELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPKLLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYCVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHDHYTQKSLSLSPGK

SEQ ID NO: 45 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLSGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 47 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRESQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYALPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWVSNGQPENNYKTTPPVLDSDGSFFLYSKLTVDKSRWQQGNVFSCSVMHEGLHNHYTQKSLSLSPGK

SEQ ID NO: 50 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSVGSFFLYSKLTVDKSRWQQGNVFSCSVMHEALHNHYTQKSLSLSPGK

SEQ ID NO: 51 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVGKSRWQQGNVFSCSVMHEGLHNHYTQKSLSLSPGK

SEQ ID NO: 52 - protein, 325 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 54 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 55 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMLEGLHNHYTQKSLSLSPGK

SEQ ID NO: 56 - protein, 395 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 57 - protein, 326 aa, Artificial Sequence
  Synthetic Construct
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSLFLYSKLTVDKSRWQQGNVFSCSVMHEALHNHYTQKSLSLSPGK

SEQ ID NO: 58 - protein, 106 aa, Artificial Sequence
  Synthetic Construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC